(19) United States
(12) Patent Application Publication
(10) Pub. No.: US 2010/0022987 A1
(43) Pub. Date: Jan. 28, 2010

(54) MEDICATION DELIVERY SYSTEM

(76) Inventors: Walter John Bochenko, Encinitas, CA (US); Theodore Craig Chan, San Diego, CA (US); Shawn Wayne DeKalb, San Diego, CA (US); James Patrick Killeen, San Diego, CA (US); Douglas Arthur Palmer, San Diego, CA (US); Michael J. Sohmer, Spring Valley, CA (US); Gary Michael Vilke, San Diego, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/509,271

(22) Filed: Jul. 24, 2009

Related U.S. Application Data
(60) Provisional application No. 61/083,213, filed on Jul. 24, 2008.
(60) Provisional application No. 61/088,768, filed on Aug. 14, 2008.
(60) Provisional application No. 61/093,436, filed on Sep. 1, 2008.

(51) Int. Cl. A61M 5/31; A61M 25/16; A61M 5/20

(52) U.S. Cl. 604/500; 604/533; 604/134; 604/246; 604/140

(57) ABSTRACT

A medication delivery system includes an array of cartridge-receiving ports, a medication receiving fluid conduit, and a plurality of dispensing elements. Each port includes a fluid channel terminating at a fluid outlet and is configured to receive one of a plurality of cartridges that each contain a bolus volume of medication. The medication-receiving fluid conduit is fluidically coupled to the fluid outlet of each cartridge-receiving port at a first region and to a system outlet conduit at a second region (which in turn can be coupled to a patient catheter line). Each dispensing element corresponds to a single cartridge-receiving port so that, upon activation of the dispensing element, at least a portion of the bolus volume of medication flows through the fluid outlet of each cartridge-receiving port into the medication-receiving fluid conduit. Related apparatus, systems, and methods are also disclosed.

### REFERENCE TO PRIORITY DOCUMENTS

This application claims priority of U.S. Provisional Patent Application Ser. No. 61/083,213, entitled “System for Emergency Medication Administration,” filed Jul. 24, 2008; and 61/088,768, entitled “System for Emergency Medication Administration,” filed Aug. 14, 2008; and 61/093,436, entitled “Versatile System for Emergency Medication Administration,” filed Sep. 1, 2008. Priority of the filing dates of Jul. 24, 2008, Aug. 14, 2008, and Sep. 1, 2008, are hereby claimed, and the disclosures of the Provisional Patent Applications are hereby fully incorporated by reference.

### BACKGROUND

Therapeutic treatments can sometimes involve parenteral delivery of a variety of medications according to protocols that require specific timing and dosages. Such complex protocols of medical infusions can cause confusion by healthcare workers and lead to treatment mistakes. Life-threatening medical emergencies, for example, can require complicated protocols involving the injection of multiple potent and potentially dangerous medications. An example of such a critical medication decision and action sequence is the ACLS (Advanced Cardiac Life Support) protocol set forth by the American Heart Association (AHA). This protocol is complicated, requiring even experienced emergency physicians to carry booklets with them in order that they can refer to them during cardiac emergencies. This is complemented by a “crash cart” that contains supplies that are used during an emergency and includes a drawer full of syringes and bottles of medications used during the ACLS protocol. The use of booklets and crash carts with the need to open boxes, prepare syringes and draw up specific doses of medications, while helpful, still offers opportunities for delay and error.

Access to the medications themselves can also cause delay and error during the administration of a complex treatment protocol. To administer medications to a patient, healthcare workers need to locate the various medications, remove them from their cardboard containers, assemble the syringes, uncap the needles, draw the proper dosage of medication into the syringe, and inject the dose into a patient fluid line while under sterile technique. The process wastes precious time, is prone to dosing errors, and increases the risk of needle sticks to the healthcare workers.

### SUMMARY

Disclosed herein is a medication delivery system including an array of cartridge-receiving ports for receiving one of a plurality of cartridges, each cartridge containing a bolus volume of medication. Each port has a fluid channel terminating at a fluid outlet. The system also includes a medication-receiving fluid conduit fluidically coupled to the fluid outlet of each cartridge-receiving port at a first region and to a system outlet conduit at a second region. The system outlet conduit is adapted to be coupled to a patient catheter line. The system also includes a plurality of dispensing elements, each corresponding to a single cartridge-receiving port. Upon activation of the dispensing element, at least a portion of the bolus volume of medication flows through the fluid outlet of each cartridge-receiving port into the medication-receiving fluid conduit.

The system can also include a plurality of cartridges, each cartridge having a hollow outer casing having a proximal end and a distal end with an internal cavity extending therebetween, a plunger. The bolus volume of medication can be contained within the internal cavity of the casing. The outer casing of each cartridge can be translatable in a first direction toward each cartridge-receiving port. The plunger of each cartridge can include a fluid outlet and each cartridge-receiving port further include a fluid inlet. Each cartridge fluid outlet can fluidically couple to each port fluid inlet such that the bolus volume of medication is in fluid communication with the medication-receiving fluid conduit. Each dispensing element, when activated, can urge at least a portion of the bolus volume of medication to flow into the medication-receiving fluid conduit toward the system outlet conduit.

The volume of medication in the plurality of cartridges can be epinephrine, lidocaine, amiodarone, atropine, magnesium sulfate, naloxone, adenosine, sodium bicarbonate, dextrose, vasopressin, and calcium chloride. The system can deliver sequential volumes of medication to a patient according to an advanced cardiac life support protocol.

At least a portion of each of the plurality of cartridges can be disposed within a portable first cassette that fluidically couples each of the cartridges to a single cartridge-receiving port upon installation of the first cassette into the system. The cassette can include a trailing end and a leading fluid delivery end relative to a direction of installation into the system. The trailing end can be positioned higher than the leading fluid end when the first cassette fluidically couples each of the cartridges to the single cartridge-receiving port. The first cassette can have dimensions defined along three mutually perpendicular axes including a columnar axis, a transverse axis, and a displacement direction axis. Each cartridge can be disposed within the first cassette along the columnar axis of the first cassette.

The system can further include a plurality of medication-receiving fluid conduits fluidically coupled to a plurality of cartridge-receiving ports. The plurality of medication-receiving fluid conduits can be fluidically in parallel with one another. The plurality of cartridges can be disposed within a plurality of cassettes. The plurality of cassettes can fluidically couple each of the cartridges to a single cartridge-receiving port upon installation of the plurality of cassettes into the system. Each of the plurality of cassettes can be installed into the system in a side-by-side configuration such that the cassettes are compactly arranged in a housing of the system along the transverse axis. The plurality of cartridges can be fluidically coupled to the plurality of medication-receiving fluid conduits in at least two dimensions.

The system can further include a sleeve axially disposed within each cartridge-receiving port. The sleeve can apply a restraining force on the plunger in a second direction opposite of the first direction upon translation of the outer casing in the first direction. Translation of the outer casing in the first direction and restraint of the plunger from translation in the first direction can reduce a volume of the internal cavity. Each of the dispensing elements can translate the outer casing of each cartridge in the first direction. The dispensing element can be a compressed spring, a source of compressed air, or a push rod. The plunger of the system can be located near the proximal end of the casing and a distance away from the distal end of the casing. The bolus volume of medication can be disposed inside the internal cavity between the plunger and the distal end of the casing. Each dispensing element can translate the plunger of each cartridge in the first direction toward the distal end of the casing.

The system can also include a plurality of activation elements, each activation element being mechanically associated with a single dispensing element and when activated, triggers the dispensing element to dispense at least a portion of the bolus volume of medication from the internal cavity. The system can also include a controller wherein the plurality of activation elements are selectively activated by the controller to trigger the dispensing elements to dispense at least a portion of the bolus volume of medication from the internal cavity.

The system can also include a fluid supply line to deliver a pressurized supply of fluid from a fluid source to the first and second medication-receiving conduits. The plurality of medication-receiving conduits each can include a one-way valve that controls forward and backward fluid flow facilitating selective flushing of each medication-receiving conduit. The system can further include a one-way valve disposed in each cartridge-receiving port that allows flow from each cartridge into the medication-receiving conduit and prevents flow from the medication-receiving conduit into each cartridge.

The system can further include a plurality of sensors disposed within each cartridge-receiving port. Each sensor can detect status information of a single corresponding cartridge, the status information detected selected from the group comprising presence of each cartridge coupled to a single port, proper coupling of a cartridge to a single port, volume of medication within each cartridge, and type of medication within each cartridge. The system can also include a sensor disposed within a housing of the system. The sensor can detect status information of the first cassette upon installation of the first cassette into the system, the status information detected selected from the group comprising presence of the cassette installed into the system, proper installation of a cassette, and presence of a cartridge disposed within the cassette.

The system can further include a fluid flow metering device disposed within the medication-receiving fluid conduit between the first region and the system outlet conduit. The fluid flow metering device can include a controller selectable between a bolus mode and a controlled mode that controls the flow of the volume of medication through the system outlet conduit. The bolus mode can deliver all of the bolus volume of medication contained within the internal cavity to the system outlet conduit. The controlled mode can deliver a volume of medication to the system outlet conduit that is less than the bolus volume of medication contained within the internal cavity of the casing.

Also disclosed herein is an interrelated medication delivery system including a medication-receiving fluid conduit having a plurality of medication-receiving ports to fluidically communicate with a corresponding plurality of cartridges, each cartridge containing a bolus volume of medication. The system also includes a metering system having an inlet port in fluid communication with the medication-receiving fluid conduit, an outlet port in fluid communication with a patient catheter line, and a dosage metering device. The system also includes a controller selectively operable in one of two modes including a controlled mode wherein the metering system meters a prescribed portion of the bolus volume of medication to the outlet port, and a full bolus mode wherein the metering system meters the bolus volume of medication to the outlet port.

The metering system can further include a first conduit positioned between the inlet port and the outlet port. The system can be operable in the controlled mode and deliver a first portion of the bolus volume of medication through the medication-receiving fluid conduit to the outlet port and a second portion of the bolus volume of medication to the first conduit. The first portion can be delivered to the outlet port after the second portion is delivered to the first conduit. The first conduit can terminate at a waste container or an alternate reservoir.

The system can further include a plurality of cartridges. Each cartridge can includes a hollow outer casing having a proximal end and a distal end with an internal cavity extending therebetween, a plunger, and a bolus volume of medication contained within the internal cavity of the casing. The system can include a medication-receiving fluid conduit that fluidically couples to the plurality of cartridges through corresponding medication-receiving ports.

Also disclosed herein is an interrelated method of medication delivery. The method includes providing a system having a medication-receiving fluid conduit including a plurality of cartridge-receiving inlet ports at a first region and a fluid outlet at a second region. The ports are mechanically supported on a rigid base in a housing of the system. The method also includes installing into the system a cassette containing a plurality of cartridges each having a volume of medication disposed therein. Installing the cassette couples each cartridge with a single cartridge-receiving inlet port. The method also includes coupling the fluid outlet to a patient catheter line and activating a dispensing element corresponding to at least one of the plurality of cartridges. The method also includes displacing at least a portion of the volume of medication from that at least one cartridge, the volume of medication flowing into the medication-receiving fluid conduit and through the fluid outlet.

The subject matter described herein provides many advantages. For example, the current subject matter allows for the rapid delivery of medications, such as the ACLS protocol, with less delay and probability for error so that doctors and other medical specialists can quickly and safely focus on patient symptoms and responses rather than the details of administering the protocol. The current medication delivery system improves the reliability, speed, accuracy, and ease of administering, such as in emergency situations.

More details of the devices, systems and methods for delivering medications are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings. Generally speaking the figures are not to scale in absolute terms or comparatively but are intended to be illustrative of claimed features. Also, relative placement of features and elements may be modified for the purpose of illustrative clarity.

FIG. 1 is a block diagram of a drug delivery system.

FIG. 2A is a block diagram of a system as in FIG. 1.

FIG. 2B is a block diagram of a system as in FIG. 1 including a dosage metering system.

FIG. 2C is a block diagram of a system as in FIG. 1 using a dosage metering system with an alternate fluid reservoir.

FIG. 3A is a schematic diagram of a top view of a cassette containing ten cartridges for use with the drug delivery system. The cartridges are shown arranged along a columnar axis C and each cartridge is shown having a long axis parallel to axis D.

FIG. 3B is a schematic diagram of a front view of three cassettes arranged along a transverse axis T with each cassette having an arrangement of cartridges arranged along a columnar axis C.

FIGS. 4A, 4B, and 4C are top, front and side views, respectively, of a drug delivery system illustrating how cartridges can be loaded into system along an axis D in a direction DI (direction of installation).

FIG. 5 is a cross-sectional schematic view depicting a single cartridge installed but not yet coupled to a cartridge receiving conduit or needle.

FIGS. 6A, 6B, 6C, 6D, and 6E are cross-sectional schematic views that depict the activation of cartridge such that the cartridge is coupled to needle whereby a volume of medication held in the cartridge is transferred to medication-receiving conduit via a conduit.

FIG. 7A is a flow chart representation of an operation of a system as illustrated in FIG. 2A.

FIG. 7B is a flow chart representation of an alternate operation of a system as illustrated in FIG. 2B.

FIG. 7C is a flow chart representation of an alternate operation of a system as illustrated in FIG. 2C.

FIGS. 8A and 8B are cross-sectional schematic views that depict a cartridge and conduit assembly.

FIG. 8C is an end overlay view depicting elements of a combination of a cartridge and cartridge assembly.

FIG. 8D is an end overlay view of a conduit assembly.

FIG. 8E is an end overlay view of a cartridge.

FIGS. 9A, 9B, 9C, and 9D are cross sectional schematic views that depict the coupling of a cartridge to a conduit assembly.

FIG. 10A is a side cross sectional view of a cartridge having a disabling portion and a portion of a conduit assembly.

FIG. 10B is an overlay view depicting how disabling portion of FIG. 10A disables conduit sterility cap.

FIG. 11A is a side cross sectional view of a cartridge having a disabling portion and a portion of conduit assembly.

FIG. 11B is an overlay view depicting how disabling portion of FIG. 11A disables conduit sterility cap.

FIGS. 12A and 12B are side cross sectional views depicting how disabling a portion of cartridge disables a sleeve sterility cap and a disabling portion of sleeve disables a cartridge sterility cap.

### DETAILED DESCRIPTION

Described herein are devices, systems and methods for rapidly delivering medications, such as for the ACLS protocol, with less delay and probability for error so that doctors and other medical specialists can quickly and safely focus on patient symptoms and responses rather than the details of administering the protocol. Described herein are devices, systems and methods that deliver medication that improves reliability, speed, accuracy, and ease of administering, such as in emergency situations. The medication delivery systems described herein are of a relatively small size such that they can be easily stored and readily transported, such as on a “crash cart.” The medication delivery systems described herein can be used in a hospital or another healthcare setting such as in an ambulance, skilled nursing facility, medical clinic, and the like. The medication delivery systems described herein can be used for adult or pediatric patients. The medication delivery systems can be used with a variety of routes of administration such as parenteral routes of administration including intravenous (IV) or intraosseous (IO).

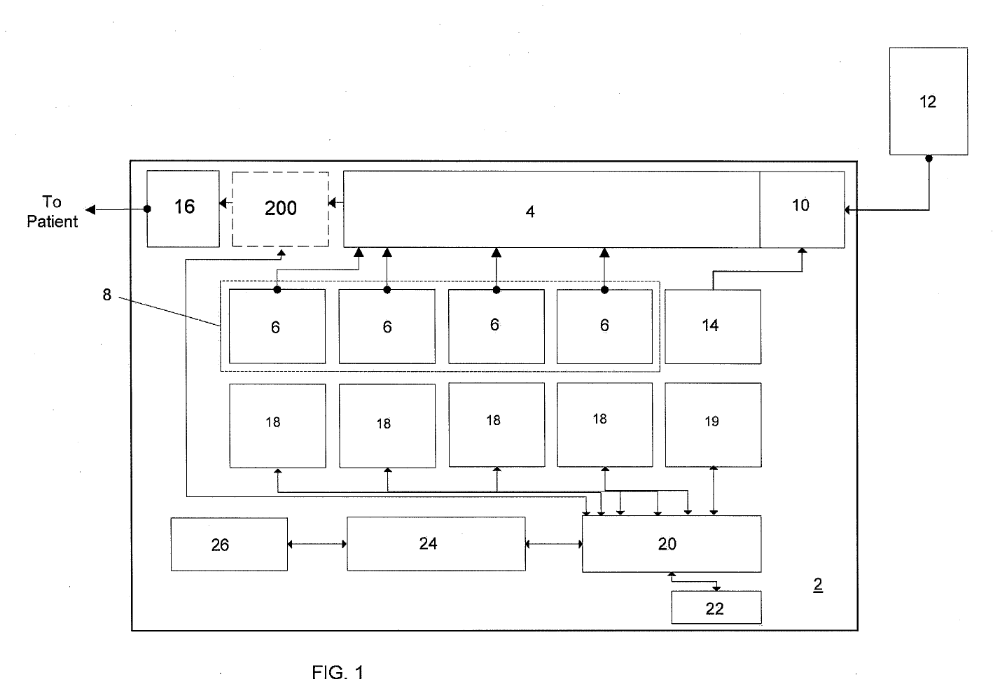

A medication delivery system is depicted in block diagram form according to FIG. 1. The system 2 includes a medication-receiving fluid conduit 4 that can receive medications from a plurality of pre-filled, medication-containing cartridges 6, for example through an array of cartridge-receiving ports. A removable cassette 8 can be used to facilitate proper installation of one or more cartridges 6 into system 2. Medication-receiving conduit 4 can be fluidically coupled to an input conduit 10 from which it receives fluid from a fluid source 12 that is external to system 2 as well as from a fluid source (not shown) that is internal to system 2. Medication-receiving conduit 4 can be coupled to an outlet conduit 16 that can deliver fluid to a patient such as through a parenteral fluid line. Fluids delivered from the fluid sources can include, for example, a saline solution or other fluid replacement solution.

The system 2 can also include a plurality of cartridge-receiving ports or cartridge interfaces 18 and a flush interface 19 that can selectively activate cartridges 6 as well as a fluid source 14 to enable medications to be selectively delivered from cartridges 6 to medication-receiving conduit 4 for the rapid delivery of bolus infusions. Each cartridge 6 at a cartridge interface 18 can be manual activated, electrically activated via controller 20 and selectively either manually or electronically activated. It should be appreciated that manual mode can be available or selected even when the system 2 is being used in an automatic mode. For example, the manual administration of a drug can be available as a back-up operating mode in the event of some kind of failure in the system. Cartridges and, if present, cartridges carried within one or more cassettes can remain in place during manual administration.

Each cartridge 6 can contain a predetermined, initial volume of medication sometimes referred hereafter as a “bolus” of medication. A cartridge 6 can inject the full bolus of medication into medication receiving conduit 4 in response to activation. The “bolus” can be equal to a maximum prescribed medication which would be given to an adult patient, for example. Delivery of the bolus can occur rapidly.

The system 2 can optionally include a “controlled bolus” dosage metering system 200 that can be internal or external to the system 2. The dosage metering system 200 can control fluid outflow in pulsed increments from the system such that aliquots of medication are delivered to the patient. Dosage metering system 200 can receive fluid from medication receiving conduit 4 and provide at least a portion of the full bolus to output conduit 16. Dosage metering system 200 can operate in at least two selectable modes: a “bolus” mode and a “controlled bolus” or metering mode. In the “bolus” mode the metering system 200 can deliver the full bolus of medication to output conduit 16. In the “controlled bolus” mode the metering system 200 can control the delivery amount of the full bolus. For example, the metering system 200 can control or meter a prescribed aliquot dosage of medication to output conduit 16. An aliquot dosage of medication can be less than the full bolus of medication contained within the cartridge 6.

Controller 20 can be coupled to I/O 22 and user interface 24 that includes display 26. I/O 22 allows external devices such as a computer to be coupled to system 2 enabling external control of system 2 and/or information to be received from or sent to system 2. User interface 24 can be used to control system 2 or to view a status of system 2 via display 26. Each cartridge interface 18 can include one or more sensors that provide status information to controller 20 indicative of a status of each cartridge 6. Such status information can be indicative of whether one or more cartridges 6 are present, whether a cartridge 6 is being activated, whether the proper cartridge is loaded, proper loading of cartridge, whether a cartridge 6 has already been activated and/or whether it has functioned properly, what drug is pre-loaded and into which cartridge, completion of medication dispense and the like. If a cartridge 6 is loaded into a cassette 8, the cassette 8 can also include one or more sensors and provide feedback information about cartridge status as well as cassette status.

Controller 20 can receive instructions that define a prescribed controlled bolus dosage of a medication from a cartridge 6 to be delivered to a patient such as over a period of time for medication delivery. The instructions can be received from user interface 24 or from I/O 22. Prescribed controlled bolus dosage can be equal to or less than the full bolus or a dosage equal to or less than the full bolus amount. In response to the instructions the controller 20 can select a “bolus” mode if the prescribed dosage desired equals the full bolus to be delivered immediately to output conduit 16. Alternatively, the controller 20 can select a “controlled bolus” metering mode if the prescribed dosage desired is less than the full bolus or if there is a need to meter an aliquot of medication over a controlled time period.

The metering system 200 can deliver the full bolus of medication from cartridge 6 if the “bolus” mode is selected. The metering system 200 can also meter a prescribed controlled bolus dosage if the “controlled bolus” metering mode is selected wherein the prescribed dosage may be less than the full bolus or it may be the full bolus that is metered over a controlled time period. The delivery rate can be selected to dispense medication over a period of time until the selected metered amount has been fully delivered to the outgoing conduit 16. Additionally, the rate for a specific medication can be non-linear in that the rate changes over a period of time.

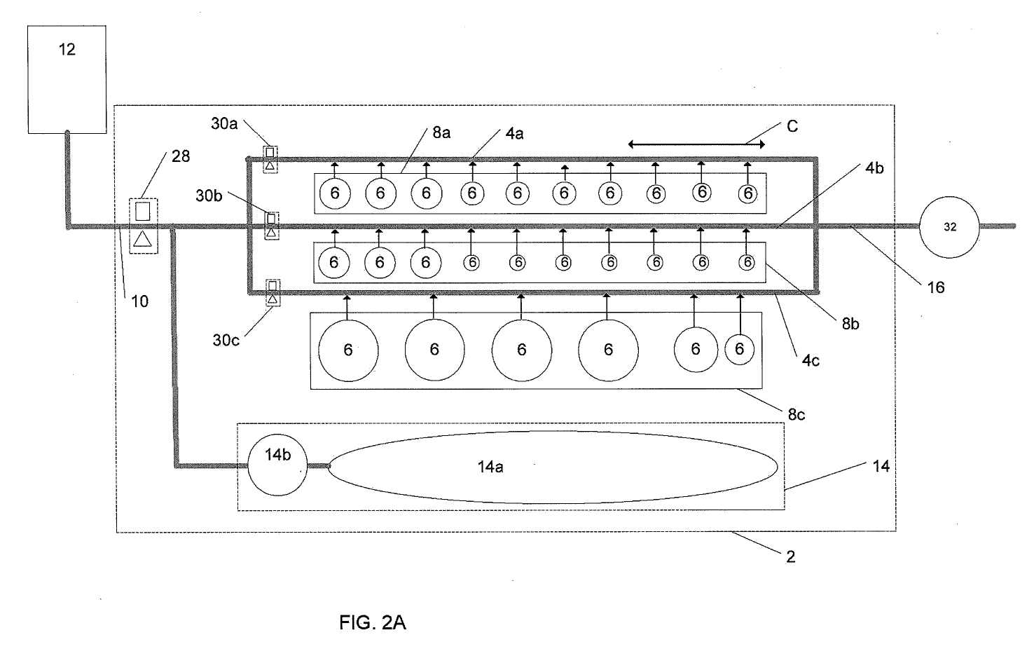

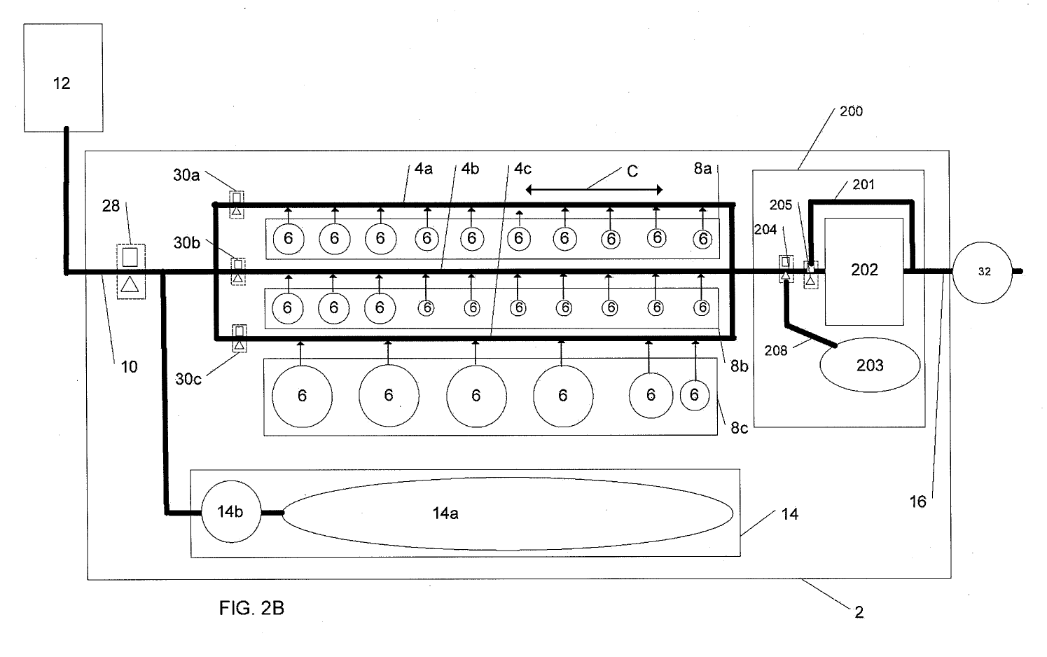

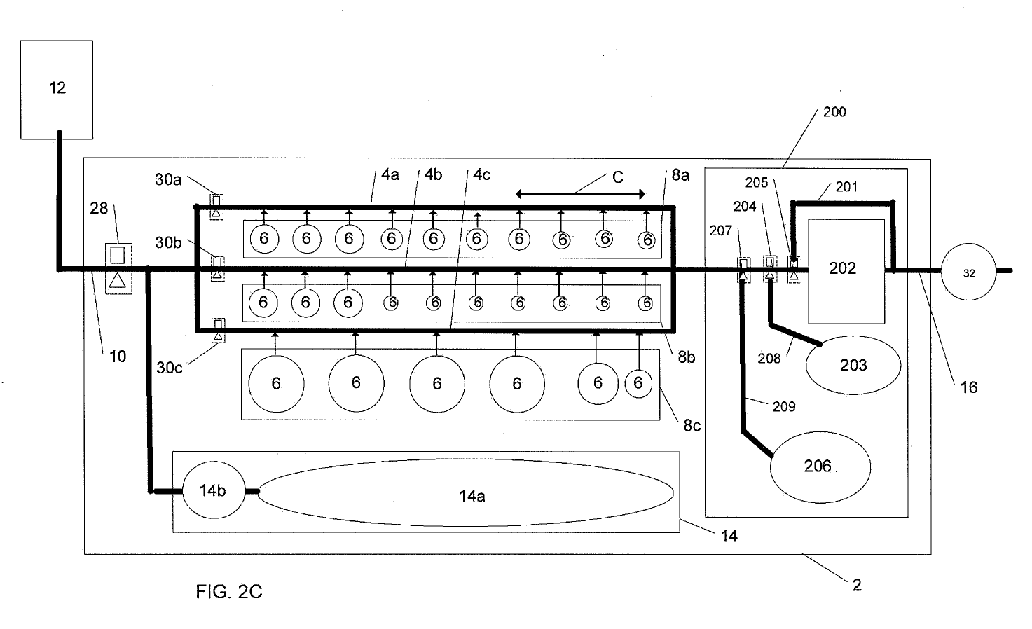

The dosage metering system 200 can also be used as a safety mechanism. Should a cartridge 6 mistakenly release an unintended bolus of medication, controller 20 can block the dose by diverting the unintended medication to a waste container 203 contained within dosage metering system 200 (see FIGS. 2B, 2C). A subsequent sequence of events such as flushing the line with a flush solution such as saline and resetting of the system can then be performed.

FIG. 2A depicts a fluid flow path of a medication system 2. Fluid from a fluid source 12, such as a fluid container or an IV bag, that arrives at input conduit 10, can pass through a system of fluidically parallel medication-receiving conduits 4 (4a, 4b, and 4c), and pass out of output conduit 16 to a patient (not shown). System 2 also can include an air filter 32 for removing air before fluid passes from output conduit 16 to the patient. Medication-receiving conduits 4 can have a branching configuration such that the medication-receiving conduits 4 branch out from input conduit 10 and carry fluid in parallel before being joined into one fluid conduit as they pass into output conduit 16. As fluid passes through medication-receiving conduits 4 the fluid can receive medication from cartridges 6 via one or more conduits as will be described in more detail below. The parallel arrangement of the conduits can provide for faster delivery of medication to a patient with a minimization of flush solution needed. The parallel arrangement of conduits also allows for the system to be more efficiently sized and compact.

FIG. 2B depicts a fluid flow path of a medication system 2 including a dosage metering system 200. Dosage metering system 200 can receive fluid from medication receiving conduits 4 and then pass fluid to output conduit 16. The system 2 can also include a bypass conduit 201 to selectively bypass the metering system 200 and conduit 208 to dispose unmetered medication to a container 203 rather than the patient. The unmetered medication can be diverted to the container 203 prior to delivery of the metered medication aliquot to the patient. Alternatively, the metered medication aliquot can be delivered to the patient prior to delivery of the unmetered medication to the container 203. Bypass conduit 201, dosage metering device 202, waste container or bag 203, and valves 204 and 205 can all be under the control of controller 20.

As mentioned previously, dosage metering system 200 can selectively operate in a “bolus” mode and a “controlled bolus” metering mode. In the “bolus” mode dosage metering system 200 can provide a bolus medication delivery by routing medication received from a cartridge 6 through a bypass conduit 201. During this mode, valve 205 can couple medication receiving conduits 4 to bypass conduit 201 which passes medicated fluid to output conduit 16. In the “bolus” mode the full dose of medication provided by cartridge 6 can be delivered to a patient. The bolus can be a maximum prescribed amount for a patient. If, however, the prescribed amount is less than this bolus amount then the metering mode can be used to set an aliquot of medication to be delivered as described below.

In the “controlled bolus” metering mode dosage metering system 200 uses dosage metering device 202 to meter a prescribed amount of medication received from cartridge 6 to output conduit 16. During this mode, valve 205 can couple the medication receiving conduits 4 to dosage metering device 202. Dosage metering device 202 can meter an aliquot of medication over a period of time or simply a rate of delivery from cartridge 6 according to the prescribed amount.

After the prescribed amount of medication has been metered by dosage metering device 202, valves 204 and 205 can operate cooperatively to discard any remaining medication from cartridge 6 and upstream fluid conduit to waste container 203. Valve 205 can close while valve 204 remains opens to allow medication to flow from cartridge 6 to waste container 203. A pump in dosage metering device 202 can transfer any remaining medication to waste container 203. The pump 14b can flush any remaining medication to waste container 203. In other words, the full bolus volume of the medication can be evacuated from the cartridge and the non-prescribed portion can be discarded into a waste container or other reservoir. As a safety feature, the fluid can also pass first to the container 203 to deliver an unwanted portion of the medication from cartridge 6 and then deliver the remaining prescribed dosage aliquot of medication to the patient.

As mentioned, the flush pump 14b can be used to flush remaining medication to waste container 203. The type of pump can vary. Pumps can include positive displacement pumps, peristaltic pumps, diaphragm pumps, syringe pumps, positive pressure bag squeezing pumps, pressurized gas pumps, or other type of parenteral mediation delivery pumps. The pump can be under control of controller 20.

The system 2 can also include a pump that controls flow of medication from the cartridge to the patient. In an example, the system 2 can include a reciprocating piston pump, or other pump as mentioned previously, that could be positioned downstream of the medication cartridge. The pump can be self-metering in that it pushes a known amount of fluid into the medication-receiving conduit 4 with each piston stroke. Each port position for each cartridge loaded into the system could include a separate pump and piston.

FIG. 2C depicts a medication system 2 that is similar to the system depicted in FIG. 2B except that it further incorporates an alternative reservoir or container 206, valve 207, and conduit 209. Except for the added container 206 and valve 207 and conduit 209, elements of FIG. 2C have a similar function as those elements depicted with respect to FIG. 2B. Unmetered medication can be selectively routed via valve 207 and conduit 209 to the container 206 for later use. Alternative reservoir 206 can receive the remainder of a bolus of medication from a cartridge 6 that can be later metered to output conduit 16. This remainder of the bolus can be retained in reservoir 206 for later use. As a safety feature, the fluid can also pass first to the reservoir 206 to deliver an unwanted portion of the medication from cartridge 6 and then deliver the remaining prescribed dosage aliquot of medication to the patient. Retention of the remaining medication provides a back-up volume of medication for use and allows for multiple injections from a single cartridge 6. For example, if a particular medication is needed in a large enough volume the cartridge containing that medication can become depleted during the protocol. The reservoir volume of medication can act as a contingency source of medication for use in a particularly long procedure requiring multiple administrations, for example ACLS procedure on a pediatric patient.

The system provides a plurality of single-dose medication packages that can be compactly housed in an array of cartridges that facilitate ease of proper loading within the system. Cartridges 6 can be carried in cassettes 8 (8a, 8b, and 8c). The cassette 8 can be a housing having multiple support features that position, support and align along one or more axes the plurality of cartridges 6 for loading together into the system 2. Cassettes 8 can facilitate proper organization and loading of cartridges 6 so that cartridges 6 do not need to be individually loaded in the system and connected to each corresponding medication-receiving conduit in the system. Cassettes 8a, 8b, and 8c can be individually associated with corresponding medication-receiving conduits 4a, 4b, and 4c, respectively. Upon being loaded into system 2, cassette 8a, 8b, and 8c can selectively inject medication from cartridges 6 into medication-receiving conduit 4a, 4b and 4c, respectively. The plurality of cartridges 6 loaded into one or more cassettes 8 facilitates the speed and ease of administering a large number of medications to a patient. The cassettes allow for a rapid and accurate association of each cartridge to a specific medication-receiving conduit and maintain an economical, compact design with respect to size of the overall system.

Each cassette 8 (one of 8a, 8b, 8c) can have a columnar dimension defined along a columnar axis C. Cartridges 6 within a cassette 8 can be generally arranged in a columnar group also along columnar axis C. A portion of medication-receiving conduit 4 that receives medication from cassette 8 can generally pass fluid from input conduit 10 to output conduit 16 along or parallel to columnar axis C according to the system depicted in FIG. 2.

While three cassettes 8a-8c are shown in the figures, more than or less than three cassettes 8 are considered herein or to distribute or carry cartridges 6 among cassettes 8 differently. For example, more than one cassette 8 can hold cartridges 6 for a given medication-receiving conduit 4. As another example, cassettes 8 can be aligned generally perpendicular to the flow path of fluid through medication-receiving conduits 4 such that a single cassette delivers fluid to more than one medication-receiving conduit 4. In yet another example, cassettes can be aligned in a variety of axes to facilitate a compact and effective means for incorporating a multitude of cassettes to deliver fluid from multitude cartridges to more than one medication-receiving conduit 4.

When medication has been injected or dispensed by a cartridge 6 into a medication-receiving conduit 4, pressurized fluid solution source 14 can provide a pressurized fluid into input conduit 10 to deliver the medication to output conduit 16 and to a patient (not shown). Fluid source 14 can include a film bag reservoir 14a containing a fluid such as saline and a peristaltic pump 14b. A valve 28 can be used to prevent backflow of fluid when pump 14b is activated or when cartridge 6 is delivering medication. Valve 28 can be, for example, a one-way valve or check valve that allows forward fluid flow from external fluid source 12 and through conduit 10 toward medication-receiving conduits 4 but resists backflow. The configuration of valve 28 can vary, for example, the valve can be a one-way check valve such as a ball, diaphragm, duck bill, swing, clapper, stop-check, lift-check, double check, double ball, pressure differential valve and other valves known in the art. Valve 28 can also be a controllable valve. Each medication-receiving conduit 4 (4a, 4b, 4c) can have an associated valve 30 (30a, 30b, 30c respectively) that prevents backflow when medication is being injected into conduit 4 (when a cartridge 6 is delivering medication) and to selectively leave open only one conduit (that just received medication) when pump 14b is activated. The valves controlling the branched fluid conduits can provide for better flushing and also renders the system fluid-tight and ensures the rapid delivery of medication to a patient.

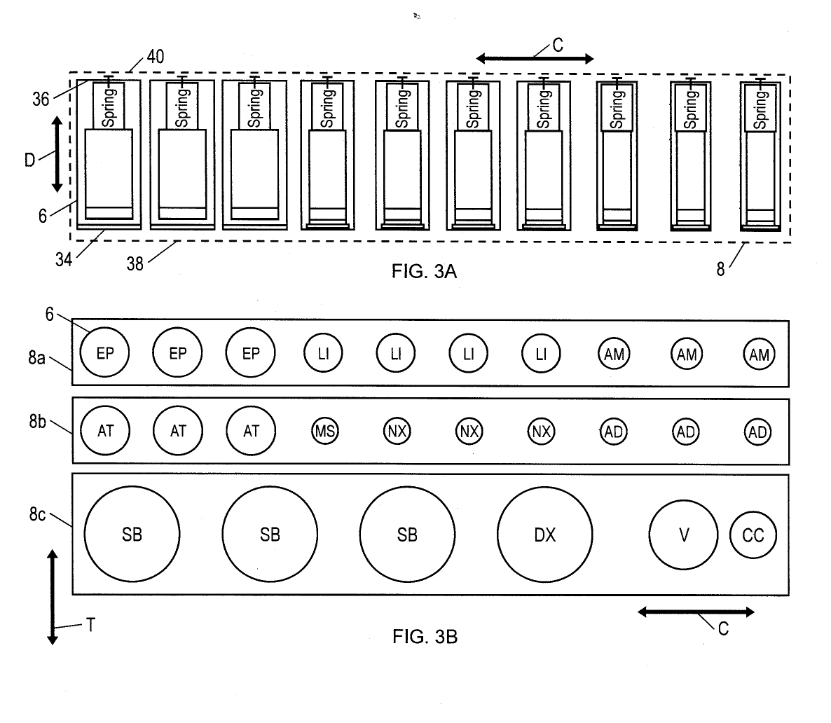

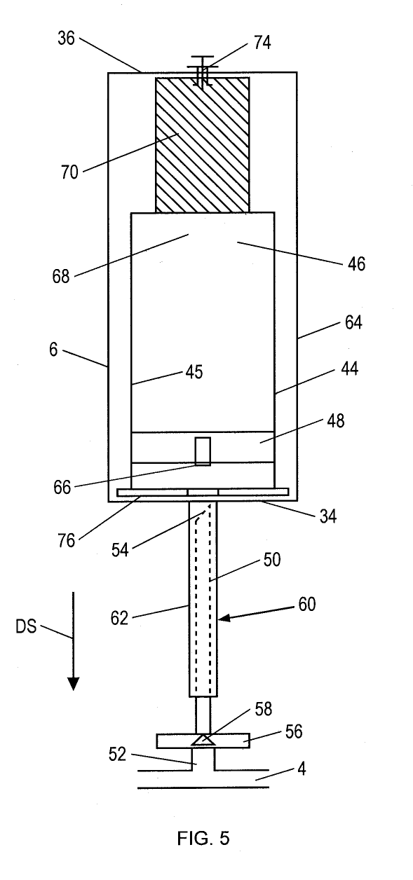

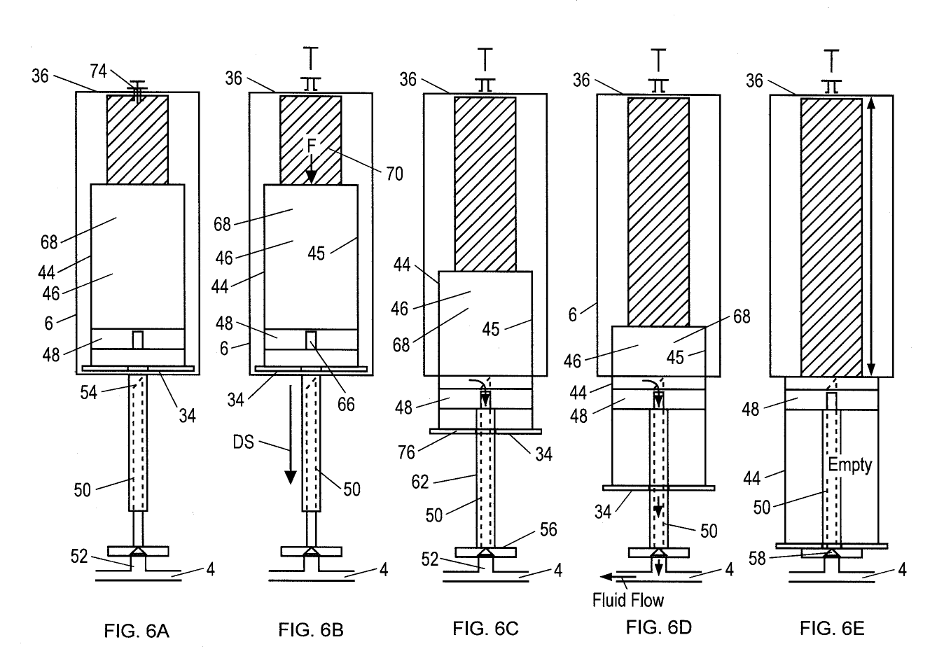

FIGS. 3A and 3B depict examples of cassettes 8 each containing a columnar group of cartridges 6. The construction and operation of cartridges 6 in system 2 will be described again with respect to FIGS. 5 and 6. Cassettes 8 (8a, 8b, and 8c) each can have outer dimensions that are defined along three mutually perpendicular axes including a columnar axis C, transverse axis T, and displacement axis D. The depicted cassettes 8a, 8b, and 8c can be arranged along the transverse axis T.

The three cassettes depicted can include a plurality of medications. The plurality of medications can be selected based on an ACLS (Advanced Cardiac Life Support) protocol. Medications can include, for example, (labeling of cartridges in figure indicated in parenthesis) epinephrine (EP), lidocaine (LI), amiodarone (AM), atropine (AT), magnesium sulfate (MS), naloxone (NX), adenosine (AD), sodium bicarbonate (SB), dextrose (DX), vasopressin (V), and calcium chloride (CC). More or less can be included in more or less than, for example, three cassettes. Variations of these or other medications for an ACLS protocol which may change from time to time in the clinical practice of emergency medicine are considered. Medications can also include emergency medications for purposes other than ACLS and/or cardiac-related emergencies. Other protocols could include protocols for use in anesthesia, procedural sedation, rapid intubation, and treatment of stroke. Table 1 below provides dosages and drug inventories of an ACLS protocol. It should be appreciated that the drug inventories and dosages are illustrative and are not meant to be limiting.

TABLE 1

| | | | | Comments |
|---|---|---|---|---|
| | | | Drug Inventory | (recommendation for ACLS |
| | Drug | Concentration | (# of pre-filled syringes) | admin.) |
| 1 | Epinephrine HCl | 1 mg/10 mL | 3-10 mL PFS | IV Push |
| 2 | Atropine Sulfate | 1 mg/10 mL | 3-10 mL PFS | IV Push |
| 3 | Vasopressin | 20 units/mL | 1-20 mL PFS | IV Push |
| 4 | Lidocaine HCl | 100 mg/5 mL | 4-5 mL PFS | IV Push |
| 5 | Amiodarone | 150 mg/3 mL | 3-3 mL PFS | IV Push |
| 6 | Naloxone | 2 mg/2 mL | 3-2 ml PFS | IV Push |
| 7 | Adenosine | 6 mg/2 mL | 3-2 mL PFS | IV Push |
| 8 | Sodium Bicarbonate | 50 mEq/50 mL | 3-50 mL PFS | IV Push 50 mL over 1-2 |
| | | | | minutes |
| 9 | Dextrose | 50% | 1-50 mL PFS | IV Push |
| 10 | Calcium Chloride | 1 gm/10 mL | 1-10 mL PFS | 10 mL |
| | | | *Rate limit 5 mL/min × | |
| | | | 2 min | |
| 11 | Magnesium sulfate | 1 gm/2 mL | 1-2 mL PFS | IV Push 2 mL over 5 minutes |
| 12 | Sodium Chloride | Normal Saline | Priming volume + 25 | Flush aliquot <10 mL |
| | | 0.9% | flushes | |
| | | | @ <8 mL/flush = 250 mL | |

Cartridges 6 can be arranged in a columnar arrangement along columnar axis C within each cassette 8. Each cartridge 6 can have a longest dimension that is aligned with displacement axis D, which is the direction that a portion of a cartridge 6 is displaced in order to inject medication into medication-receiving conduit 4 as will be depicted with respect to FIGS. 5 and 6. Each cartridge 6 can include a fluid delivery end 34 and a closed trailing end 36 with respect to a direction of installation of each cartridge into system 2. Each cassette can have a fluid delivery end 38 that is in proximity to fluid delivery ends 34 of cartridges 6 and a trailing end 40 that is in proximity to the trailing ends 36 of cartridges 6.

Cartridges 6 can also be individually installed into system 2 (without the use of cassettes). The cartridges 6 can be installed in columnar groups 8a, 8b, and 8c such that each columnar group of cartridges is aligned with columnar axis C. The columnar groups 8a, 8b, and 8c can be arranged along transverse axis T. Each cartridge can have a long axis that is parallel with displacement axis D.

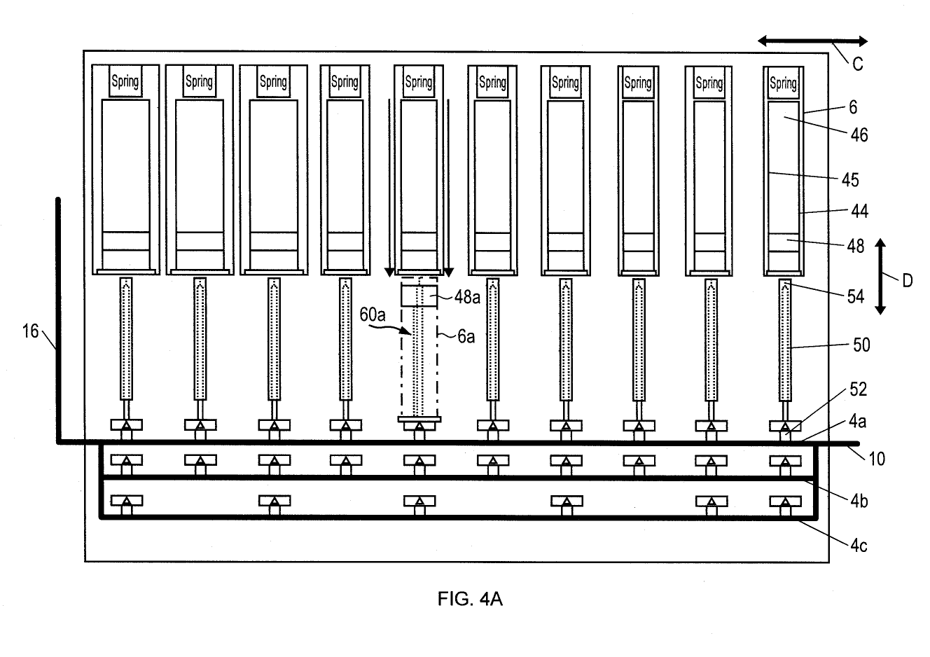

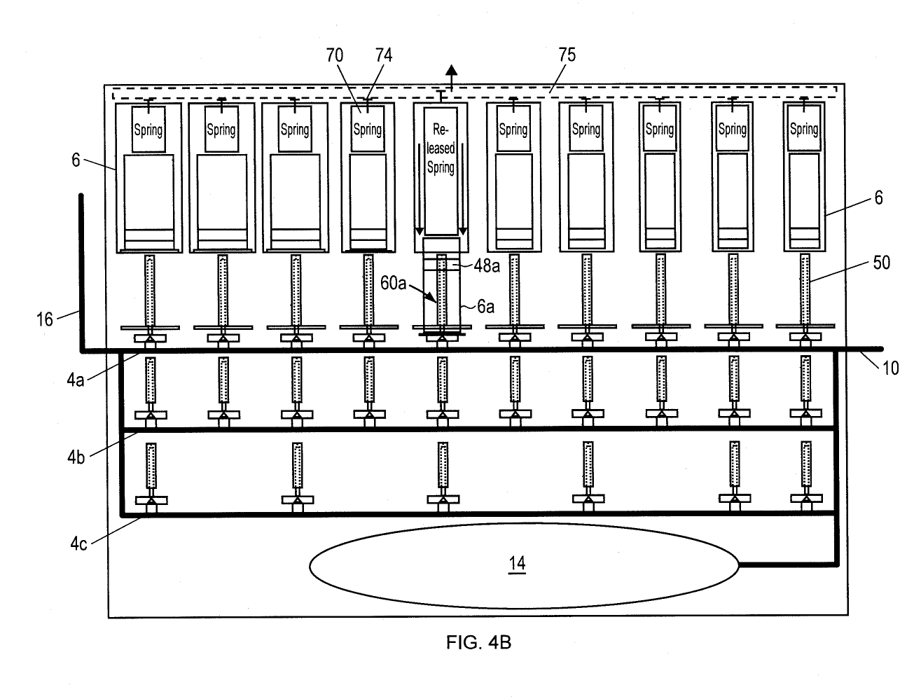

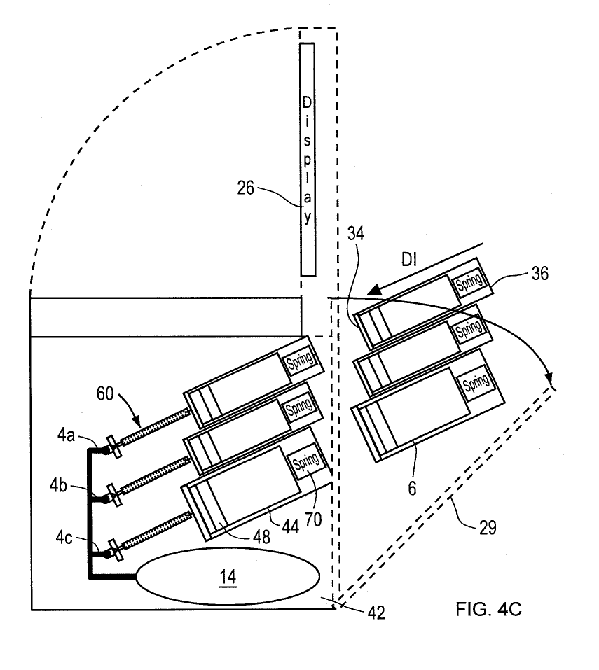

FIGS. 4A-4C are approximate schematic diagrams of medication delivery system 2 depicting the relative placement of cartridges 6 and other features in system 2 with side walls in ghost in order to depict some internal components. FIGS. 4A-4C include a top view, front view, and side view.

FIG. 4A is a top view of system 2 depicting a single row of cartridges 6 including one cartridge 6a shown in hatched lines as engaged with a cartridge-receiving conduit or connector 50a and displaced with respect to the plunger 48a such that medication would be dispensed into the medication-receiving conduit 4a. Each cartridge 6 includes a hollow cylindrical portion 44 with an internal surface 45 defining an internal cylindrical cavity 46. Contained within cavity 46 is a plunger 48. A volume of medication can be held inside cavity 46 between plunger 48 and internal surface 45. System 2 also can include a plurality of cartridge-receiving conduits 50 that are each configured to engage a cartridge 6 when a medication is selectively injected from a cartridge 6 into medication-receiving conduit 4. The cartridge-receiving conduit 50 can be a needle, such as an 18 gauge or other gauge of needle. Each cartridge-receiving conduit 50 has a proximal end 52 that can couple to medication-receiving conduit 4 and a distal end 54 that can receive medication from a cartridge 6. More details of the design and interactions between cartridges 6 and cartridge-receiving conduits 50 will be discussed with respect to FIGS. 5 and 6.

FIG. 4B is a front view of system 2 depicting similar features as those discussed above and below. System 2 includes fluid source 14. The fluid source 14 can include a supply bag 14A and pump 14b (FIG. 2B) such that when medication is injected into medication-receiving conduits 4 the medication can more quickly be pumped out of output conduit 16 and to a patient (not shown) and can separate each medication injected into medication-receiving conduit 4 to limit intermixing.

FIG. 4C is a side view of system 2 depicting the loading of cartridges 6 into system 2. A display 26 that can be flipped up from a base portion 42 of system 2 is shown in the FIG. 4C. Other types of displays 26 are considered as is known in the art. As stated previously, each cartridge 6 can have a leading fluid delivery end 34 and a trailing end 36 relative to a direction of installation (DI) of cartridges 6 into system 2. The direction of installation DI is generally parallel to the displacement axis D. The cartridges 6 can be loaded in the system 2 such that the trailing end 36 is positioned higher than the leading end 34 such that the cartridges 6 are loaded at an angle to the system 2. The angle allows entrapped air within the cartridge 6 to rise to the trailing end of the cartridge 6 and avoids delivery of air into the system or to a patient. The angle of the cartridge 6 with respect to the system can vary. The cartridges 6 can be loaded in the system 2 such as by opening a door 29 or other access feature in the base portion 42. Cassettes 8 (not shown) can each contain or be pre-loaded with a plurality of cartridges 6.

FIGS. 5, 6A-6E, 8A-E, 9A-D, 10A-B, 11A-B, and 12A-B depict details of a cartridge 6 and a corresponding conduit assembly 60. Like numerals depict like elements. It should be appreciated that although the cartridges and corresponding conduit assemblies are described in the context of reverse syringes, for example such as the ABBOJECT or BRISTOJECT, other types of fluid injection systems or syringe systems are considered. Similarly, other medicine cartridges, vials or penetrable pouches of medications can be also used in conjunction with the system. Common direct injection syringes are also considered. In this variation, a dispensing element can be used to provide a force such as air pressure or a compressed spring to drive fluid from the syringe into the fluid conduit. It should also be appreciated that fluid injections can be performed manually as well as through electronic automation. For example, manual administration such as a user exerting force on the plunger or casing or on a push rod coupled to the plunger or casing can be performed as a back-up operation mode in the event of a failure within the system. Manual administration of a drug can be readily observable by an operator and can be performed without a need for removal of a cartridge and/or cassette. For example, this can be accomplished by the opening of door 29 to access the loaded cartridges and then pushing the proximal end 36 in the displacement direction DS until the administration is completed. Further, a tubing set attached to the patient, intravenously or by interosseous routes of administration, can also include a “Y site” such that medications can be delivered directly to the patient and not through the system 2.

FIG. 5 depicts a cartridge 6 and a corresponding conduit assembly 60 including a cartridge-receiving fluid conduit 50 that extends in a first direction from a proximal end 52 to a distal end 54. Cartridge-receiving fluid conduit 50 can include a proximal end 52 that is fluidically coupled to medication receiving conduit 4. Proximate to the proximal end can be a base 56 for providing mechanical support to cartridge-receiving fluid conduit 50 and a valve 58 that prevents backflow. The valve 58 can prevent fluid from medication-receiving conduit 4 from flowing up into a cartridge 6. Cartridge-receiving fluid conduit 50 also includes a distal end 54 having a fluid inlet 54. The fluid inlet 54 can be a needle tip 54 designed to pierce plunger 48. While needle tip 54 is depicted as sharp, it can be rounded or some other shape. The needle can be an 18 gauge or other gauge of needle. Conduit assembly 60 can also include a cylindrical sleeve 62 that is axially disposed upon cartridge-receiving fluid conduit 50. The cylindrical sleeve 62 can maintain the cartridge-receiving fluid conduit 50 in a sterile condition prior to cartridge 6 displacement and to restrain motion of plunger 48 as will be discussed in more detail below.

Cartridge 6 can include an outer housing 64 surrounding a hollow cylindrical portion 44 that has an inner surface 45 defining an internal cylindrical cavity 46. A penetrable plunger 48 can be disposed within the internal cavity 46. The penetrable plunger 48 can include a feature 66 that facilitates penetration of plunger 48 by distal end 54. Contained within cavity 46 between plunger 48 and inner surface 45 can be a volume of medication 68. Feature 66 is a fluid outlet that allows fluid to flow out of the cartridge 6 and into the cartridge-receiving fluid conduit 50 before flowing into the medication-receiving fluid conduit 4. Cartridge 6 can also include a cap 76 that maintains sterility inside cartridge 6 before use. The cap 76 can be penetrable and removable. The sterility cap and sterility barriers will be described in more detail below.

Within housing 64 can be a dispensing element 70, such as a compressed spring, that can displace cylindrical portion 44 in displacement direction DS upon activation of a release mechanism 74. Release mechanism 74 is depicted in FIG. 5 as being disposed adjacent to trailing end of cartridge 36, but other locations are possible depending upon the design of release mechanism 74. For example, the release mechanism 74 can include one or more pins that contact the cylindrical portion of the cartridge 6 and mechanically prevent its displacement in the displacement direction. Displacement direction DS can be the same as the direction of installation DI. While the dispensing element 70 is depicted as located between trailing end 36 of cartridge 6 and cylindrical portion 44, it should be appreciated that other configurations are considered. For example, the dispensing element 70 can be positioned to axially surround cylindrical portion 44 and to push on a lip or other feature that is proximate to fluid delivery end 34 of cartridge 6.

The housing 64 can include other types of dispensing elements 70 or mechanisms to impart force on the cylindrical portion 44. For example the force can be automatically imparted by an electric motor, solenoid, air pressure, a compressed or expanding gas, magnetic or electromagnetic force, reciprocating piston pump, and the like. The housing 64 can include a dispensing element that can be manually depressed, or threaded etc. such that the medication within the internal cavity is evacuated from the cartridge.

FIGS. 6A-6E depict the selective injection of a medicated fluid from a cartridge 6 into a medication-receiving conduit 4 upon docking and installation of a cartridge 6 into the system 2. FIG. 6A is essentially the same as FIG. 5 depicting a state in which cartridge 6 is installed in system 2 but not yet selectively injected. According to FIG. 6B a force begins to be exerted against cylindrical portion 44 in displacement direction DS. The force is shown in FIG. 6B as being exerted by dispensing element 70. The dispensing element 70 displaces cylindrical portion 44 in displacement direction DS upon activation of a release mechanism 74 by an activation mechanism 75. As an example, a release pin can be activated by an electromechanical mechanism that is under the control of controller 20. Release mechanism 74 can be triggered or activated any number of ways such as by motor driven mechanism, a solenoid valve, switch, an air piston as well as manually activated. Also, the release mechanism 74 need not have an activation mechanism 75 and instead can be manually activated, such as by a user. It should be appreciated that the dispensing, release and activation mechanisms can all vary and that manual as well as automatically controlled mechanisms can be used.

According to FIG. 6C hollow cylindrical portion 44 can be displaced in displacement direction DS causing distal end or needle tip 54 to penetrate penetrable cap 76 and plunger 48. Cap 76 can be removed or opened prior to release of the dispensing element 70 so that needle tip 54 does not need to penetrate cap 76. Alternatively, cap 76 can be penetrable and remain in place such that needle tip 54 penetrates the cap 76 upon release of the dispensing element 70. Sleeve 62 can engage plunger 48, restraining its motion in the displacement direction DS so that cylindrical portion 44 can move relative to plunger 48 in the displacement direction DS. The relative motion of cylindrical portion 44 and plunger 48 creates a positive pressure in cavity 46 that forces the volume of medication 68 disposed in cavity 46 to begin to flow out fluid outlet 66, and through cartridge-receiving fluid conduit 50 in the displacement direction DS. Base 56 supports forces exerted on cartridge-receiving fluid conduit 50 and sleeve 62 by plunger 48 during the injection of medication from cartridge 6 into medication-receiving conduit 4.

FIG. 6D depicts continued fluid delivery of fluid out of cartridge 6 and into medication-receiving conduit 4 as the volume defined between plunger 48 and proximal region of internal cylindrical cavity 46 of the cylindrical portion 44 is reduced by the displacement of cylindrical portion 44 relative to the plunger 48 in displacement direction DS. FIG. 6E depicts a fully depleted cartridge 6 when the plunger 48 and the cylindrical portion 44 can no longer be displaced relative to each other. Valve 58 prevents backflow of fluid from medication-receiving conduit 4 into the cartridge 6.

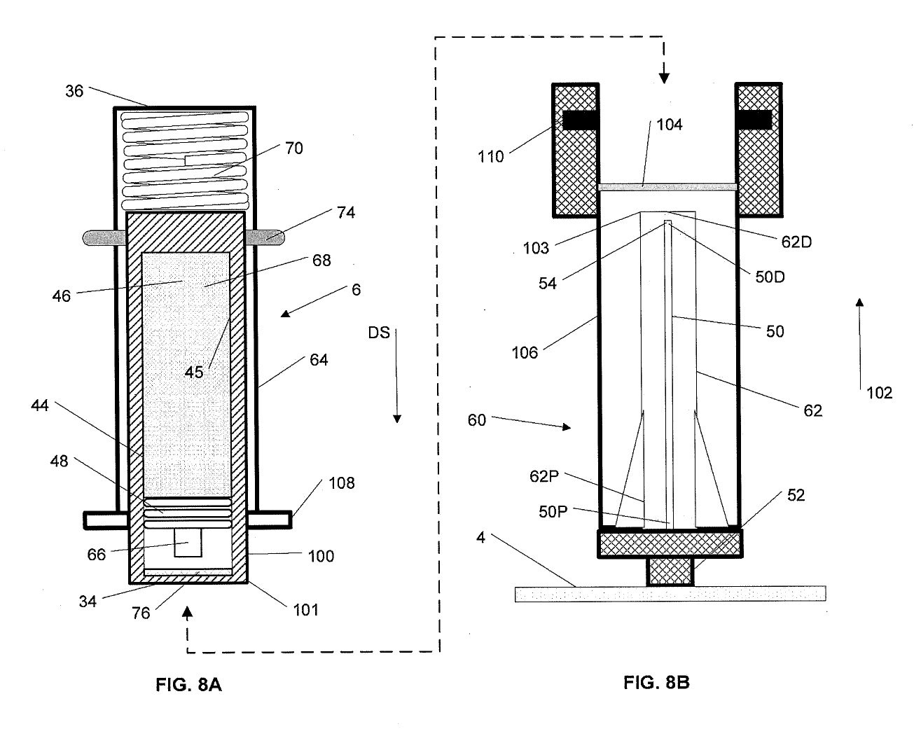

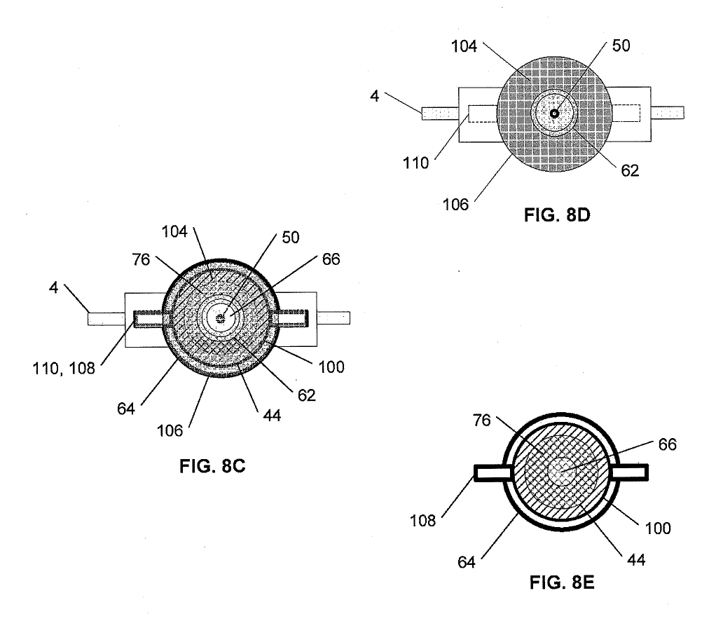

FIGS. 8A-E, 9A-D, 10A-B, 11A-B, and 12A-B depict details of a cartridge and corresponding conduit assembly that make use of one or more sterility barrier assemblies. Portions of the cartridge and also the corresponding conduit assembly can each include a sterility cap, guard or other type of barrier that maintains the sterility of the fluid outlets and inlets. These barriers can prolong the shelf-life of the cartridges by maintaining the sterility of the fluids and fluid pathways contained therein. The barriers can be penetrable, removable and sterilizable as will be discussed in more detail below. The cartridge and corresponding conduit assembly can also each include a disabling feature that can be used to penetrate the sterility barriers. In addition, the cartridge and corresponding conduit assembly depicted in FIGS. 8A-E, 9A-D, 10A-B, 11A-B, and 12A-B each include mechanical features that improve alignment and fixation between them. These features will be described in more detail below.

According to FIG. 8A, cartridge 6 can include an outer housing 64 having a cartridge mechanical feature 108. Cartridge 6 can also include a casing 100 that is slideably mounted in outer housing 64. Within casing 100 is a hollow portion 44 having an inner surface 45 defining cavity 46. Within cavity 46 is a plunger 48 having fluid outlet 66. Also within cavity 46 is a volume of medication 68 contained between plunger 48 and inner surface 45. Hollow portion 44 can be formed from glass and supported by casing 100. Casing 100 and hollow portion can also be one integral part 100 that may be formed by injection molding.

Cartridge 6 has a leading end 34 and a trailing end 36 relative to a direction of displacement DS (or direction of insertion DI) that is the direction that casing 100 is displaced to fluidically couple cartridge 6 to a corresponding conduit assembly 60. The leading end 34 of cartridge 6 can be the same as the leading end 34 of casing 100 relative to DS. Casing 100 can include a conduit sterility cap disabling feature 101 that is proximate to the leading end 34 to be discussed below. Feature 101 can also define the leading end 34.

Disposed proximate to the leading end 34 is a cartridge sterility cap 76 that maintains the fluid outlet 66 in a sterile condition prior to the coupling of cartridge 6 to conduit assembly 60. Together the cartridge sterility cap 76 and cylindrical portion 44 (casing 100) provide a cartridge sterility barrier for fluid outlet 66. The configuration of the cartridge sterility cap 76 can vary. The cartridge sterility cap 76 can be disposed in a recessed position relative to the leading end 34. The cap 76 can be a plug-type device having a penetrable, central region spanning the plug. The cartridge sterility cap 76 can be integrated with the casing 100. Alternatively, the cap 76 can be a separate device, for example, one that can be removably coupled to or mounted in or on a portion of the casing 100. The cap 76 can include a center spanning region that includes a penetrable, membrane-type barrier. In this example, the cap 76 can include a penetrable material coupled to a support ring that fits on, in or around a portion of the casing 100 such that the penetrable material spans the cavity 46.

According to FIG. 8B, conduit assembly 60 can extend away from medication-receiving fluid conduit 4 in a first direction 102 that is opposing or opposite to displacement direction DS when cartridge 6 is installed into conduit assembly 60. Conduit assembly 60 can include a cartridge-receiving conduit 50 that extends in the first direction 102 from a proximal end 50P to a distal end 50D. The cartridge-receiving conduit 50 can be a needle, such as an 18 gauge or other gauge of needle. A fluid inlet 54 is disposed proximate to or at the distal end 50D of cartridge-receiving conduit 50, which is fluidically coupled to medication-receiving conduit 4.

Surrounding the cartridge-receiving conduit 50 can be a sleeve 62 that extends in the first direction 102 from a proximal end 62P to a distal end 62D. The distal end 62D of sleeve 62 can engage plunger 48 to align fluid outlet 66 to fluid inlet 54 when cartridge 6 is coupled to conduit assembly 60. Sleeve 62 can also include a cartridge sterility cap disabling feature 103 proximate to distal end 62D. Feature 103 can define the distal end 62D.

Surrounding sleeve 62 can be a conduit housing 106. Disposed upon conduit housing 106 can be a conduit sterility cap 104. Together conduit housing 106 and conduit sterility cap 104 form a sterility barrier that preserves the sterility of cartridge-receiving conduit 50. The configuration of the conduit sterility barrier 104 can vary. The conduit sterility cap 104 can be disposed in a recessed position of the housing 106. The cap 104 can be a plug-type device having a penetrable, central region spanning the plug. The conduit sterility cap 104 can be integrated with the housing 106. Alternatively, the cap 104 can be a separate device, for example, one that can be removably coupled to or mounted in or on a portion of the housing 106. The cap 104 can include a center spanning region that includes a penetrable, membrane-type barrier. In this example, the cap 104 can include a penetrable material coupled to a support ring that fits on, in or around a portion of the housing 106 such that the penetrable material spans the bore of the housing 106.

The sterility barriers and sterility caps described herein can be manufactured of a variety of materials. They can be sterilized such as by ethylene oxide, gamma radiation or autoclave and the like and avoids fracture and aging. As such the sterility barriers and sterility caps can have a relatively prolonged shelf-life. The sterility barrier of the cartridge can be manufactured of the same material or a different material as the sterility barrier of the receiver conduit. In a variation, one or more portions of the sterility barriers can be manufactured of silicone rubber (natural rubber and synthetic), polyethylene, HDPE (high-density polyethylene), polyester or similar medical disposable material.

Conduit housing 106 can also include a conduit assembly alignment feature 110. Cartridge alignment feature 108 and conduit alignment feature 110 can be complementary mechanical features with respect to each other and enable cartridge housing 64 to be fixedly mounted to conduit housing 106. Complementary alignment features 108 and 110 also provide mechanical alignment of cartridge housing 64 with respect to conduit housing 106. Optionally, feature 110 may be located as part of the housing of system 2 so as to provide mechanical alignment and fixation of cartridge housing 64 to conduit housing 106. The configuration of the mechanical alignment and fixation of cartridge housing 64 and conduit housing 106 can vary. Features 108, 110 can have complementary shapes such that they snap together or thread together or another mechanical engagement. Features 110 and 108 can also have a lead-in design to facilitate the ease of engagement.

Cartridge 6 is configured to enable casing 100 to move or slide relative to housing 64 in displacement direction DS. A force against casing 100 can be provided by a dispensing mechanism 70 or element, for example a spring that is disposed within housing 64 can urge casing 100 in the displacement direction DS. It should be appreciated that another dispensing mechanism 70 to impart force on the casing 100 is considered such as compressed gas, expanding gas, electric motor, solenoid and the like. Manual dispensing mechanisms 70 are considered as well, for example a push rod coupled to the plunger or casing. The motion of casing 100 relative to housing 64 occurs when a release mechanism 74 is triggered such as by an activation mechanism 75 allowing dispensing mechanism 70 to displace casing 100 in direction DS relative to housing 64. The release mechanism 74 as well as the activation mechanism 75 can vary as described above. As an example, the release mechanism 74 shown in FIG. 8A can be one or more pins that contact the casing 100 and mechanically prevent its displacement in the displacement direction until released. Once released or activated, the dispensing mechanism 70 can urge the casing 100 in the displacement direction DS. The release mechanism 74 and the activation mechanism 75 can also be the same element.

FIGS. 8C-E depict portions of a cartridge 6 and conduit assembly 60 in a radial fashion. A view looking at an end of conduit assembly 60 in displacement direction DS is shown in FIG. 8D. A view in displacement direction 102 is shown of the cartridge assembly in FIG. 8E. FIG. 8C is an overlaid view of various elements of cartridge 6 and conduit assembly 60 that illustrates their relative radial positioning from the perspective of an eye looking down upon them in direction DS or 102. Some elements may be missing from each view for illustrative simplicity and clarity.

FIG. 8C depicts the combination when cartridge 6 is installed into conduit assembly 60 including medication-receiving conduit 4, hollow portion 44, cartridge-receiving conduit 50, sleeve 62, cartridge housing 64, fluid outlet 66, cartridge cap 76, casing 100, conduit cap 104, conduit housing 106, and complementary mechanical features 108 and 110. Several elements (e.g., hollow portion 44, cartridge-receiving conduit 50, sleeve 62, cartridge housing 64, fluid outlet 66, cartridge cap 76, casing 100, conduit cap 104, conduit housing 106) are circular and concentric, but it should be appreciated that other shapes are considered. Casing 100 and its associated portions are configured to fit within conduit housing 106 when casing is displaced in direction DS. Sleeve 62 can be configured to receive a fluid outlet 66 portion of plunger 48 when fluid outlet 66 is coupled to fluid inlet 54 of cartridge-receiving conduit 50.

FIG. 8D depicts portions of conduit assembly 60 including medication receiving conduit 4, cartridge-receiving conduit 50, sleeve 62, conduit sterility cap 104, conduit housing 106, and conduit mechanical feature 110. Sleeve 62 can be a cylindrical member that surrounds cartridge-receiving conduit 50. Conduit housing 106 can be a cylindrical member that surrounds sleeve 62.

FIG. 8E depicts portions of cartridge 6 including hollow cylindrical portion 44, cartridge housing 64, fluid outlet 66, cartridge cap 76, casing 100, and cartridge mechanical feature 108. Hollow cylindrical portion 44 is disposed within casing 100 and may be integral with casing 100. Elements 44 and 100 can also be the same element. Casing 100 is slideably mounted within cartridge housing 64 and is configured to slide in direction DS in order to couple fluid outlet 66 to cartridge-receiving conduit 50.

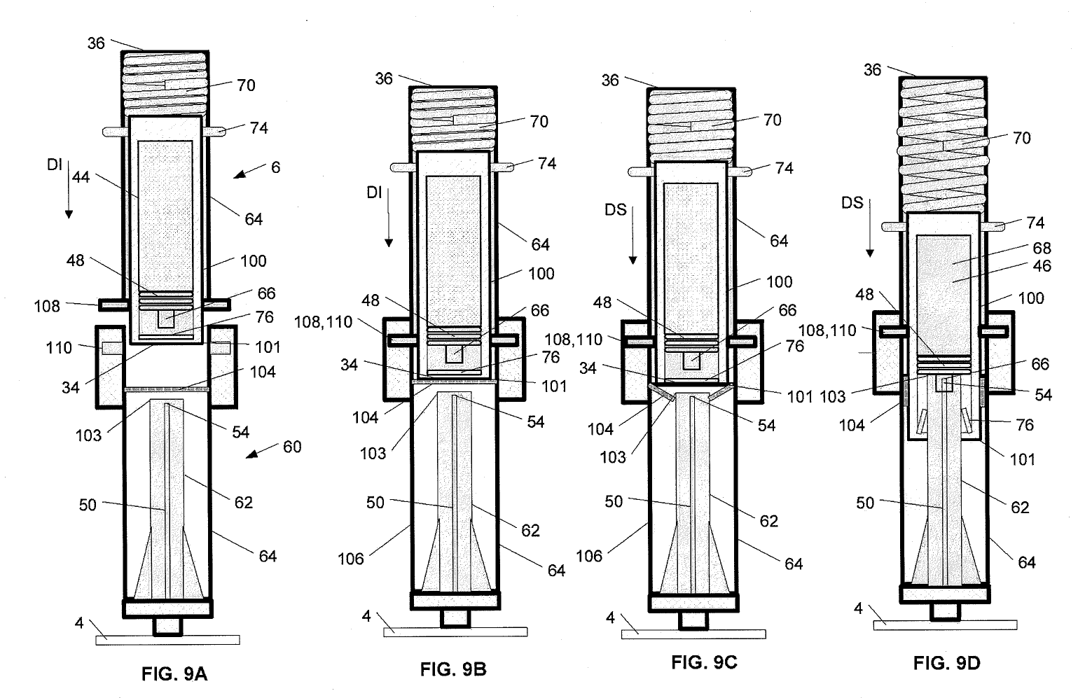

FIGS. 9A to 9D depict a sequence wherein cartridge 6 is installed upon and then fluidically coupled to the conduit assembly 60 to enable the volume of medication 68 to be transferred from cartridge 6 to medication-receiving conduit 4. FIG. 9A depicts cartridge 6 before it is installed onto conduit assembly 60. Cartridge 6 includes a leading end 34 and a trailing end 36 relative to a direction of installation DI of cartridge 6 into system 2 or onto conduit assembly 60. Cartridge 6 extends from trailing end 36 to leading end 34 in the same direction as the direction of installation DI.

FIG. 9B depicts cartridge 6 loaded into system 2 or onto conduit assembly 60. Cartridge 6 can be installed into conduit assembly 60 when cartridge mechanical features 108 inter-engage conduit mechanical features 110. Cartridge 6 can mechanically couple to conduit assembly 60 via the inter-engagement of complementary mechanical features 108 and 110. Leading end 34 of cartridge 6 is now proximate to conduit sterility cap 104.

As stated before, casing 100 can be slideably mounted in cartridge housing 64 to enable casing 100 to be displaced in displacement direction DS relative to housing 64. Release mechanism 74 can be activated by an activation mechanism 75, allowing dispensing element 70 or other energy source or force to drive or displace casing 100 in the direction DS. According to FIG. 9C, casing 100 is now displaced in displacement direction DS relative to mechanical features 108 and 110 and housing 64 and 106 that remain relatively fixed. According to FIG. 9C, disabling portion 101 has disabled the conduit sterility cap 104.

According to FIG. 9D casing 100 continues to be slideably displaced relative to fixed housing 64 and fixed complementary mechanical features 108 and 110. According to FIG. 9D, a leading edge (cartridge cap disabling feature 103) of sleeve 62 has disabled cartridge sterility cap 76. Fluid outlet 66 has coupled to fluid inlet 54 to allow the volume of medication 68 to begin flowing out of cavity 46, through fluid outlet 66, into cartridge-receiving conduit 50, and to medication-receiving conduit 4.

According to FIGS. 9C and 9D a force is exerted on casing 100 to drive casing 100 in displacement direction DS whereupon the following sequence of events takes place: (1) sterility caps 76 and 104 are disabled, (2) fluid outlet 66 is coupled to inlet 54, and (3) the volume of medication 68 flows from cavity 46 to medication-receiving conduit 4. The force has shown in FIGS. 9C and 9D is imparted by a dispensing element 70, such as a compressed spring which has been released according to the actuation of spring release mechanism 74. The dispensing element 70 driving casing 100 in direction DS can also be automatically imparted by one of an electric motor, a solenoid, or an expanding gas. The force driving casing 100 in direction DS can also be imparted manually by a mechanical device or a human finger.

Referring again to FIGS. 9A-D, it should be appreciated that the following operations [1] to [7] are illustrative and are not meant to be limiting. One or more of the operations can be excluded, added, performed simultaneously or performed in a different order. For example, the cartridge sterility cap 76 can also be sequentially disabled before conduit sterility cap 104 is disabled (operations [4] and [5] are in reverse order).
[1] Cartridge 6 is positioned for installation upon conduit assembly 60.
[2] Cartridge 6 is docketed, installed or loaded upon conduit assembly 60. Complementary mechanical features 108 and 110 align and engage.
[3] A force is exerted upon casing 100 causing casing to move in the displacement direction DS relative to conduit assembly 60.
[4] As casing 100 continues to move, conduit sterility cap 104 is disabled by a leading portion of casing 100 which may be referred to as a conduit sterility cap disabling feature 101.
[5] As casing 100 continues to move, cartridge sterility cap 76 is disabled by a leading portion of sleeve 62 referred to as cartridge sterility cap disabling feature 103.
[6] As casing 100 continues to move, fluid outlet 66 is coupled to fluid inlet 54 and a leading portion of sleeve 62 is seated against plunger 48. The sterility caps are disabled prior to coupling fluid outlet 66 to fluid inlet 54 to assure sterility of the fluid coupling.
[7] Casing 100 continues to move as the volume of medication 68 inside cavity 46 is reduced and flows into medication-receiving conduit 4.

Installation of the cartridge 6 (singly or in combination when carried within a cassette 8) into the system 2 can involve one or more multi-part operations. For example, the cartridge 6 can dock with its corresponding port or conduit assembly 60 such that the cartridge 6 is installed or in a “docked” mode with the system 2. Docking can occur without a fluid connection being formed between cartridge 6 and the conduit assembly 60 and without breach of either of the penetrable sterility barriers, if used. The cartridge and conduit assembly in the docked mode can remain sterile and can be reused if further activation is not performed. The cartridge and conduit assembly in the docked mode can also be mechanically coupled together through the coupling of their respective alignment features, if present. The cartridge 6 once docked with the conduit assembly 60 in the system 2 can then be selectively displaced in the displacement direction DS such that a fluid connection between the cartridge and the conduit assembly 60 is created. Upon displacement, one or more of the penetrable sterility barriers, if present, can be breached and a fluid coupling between the cartridge and the conduit assembly is formed. The cartridge is in a “loaded” mode with the system. Once a cartridge is docked and loaded into the system, dispensing of the medication within the cartridge 6 can begin.

Cartridges 6 can also be inserted into the system 2 pre-coupled to the conduit assembly 60. In this example, a port or interface at the leading end of the cartridge/conduit assembly couples to a corresponding port or interface within the housing of the system 2. A multi-part operation of docking, loading and dispensing as described above can also be used in a pre-coupled cartridge/conduit assembly.

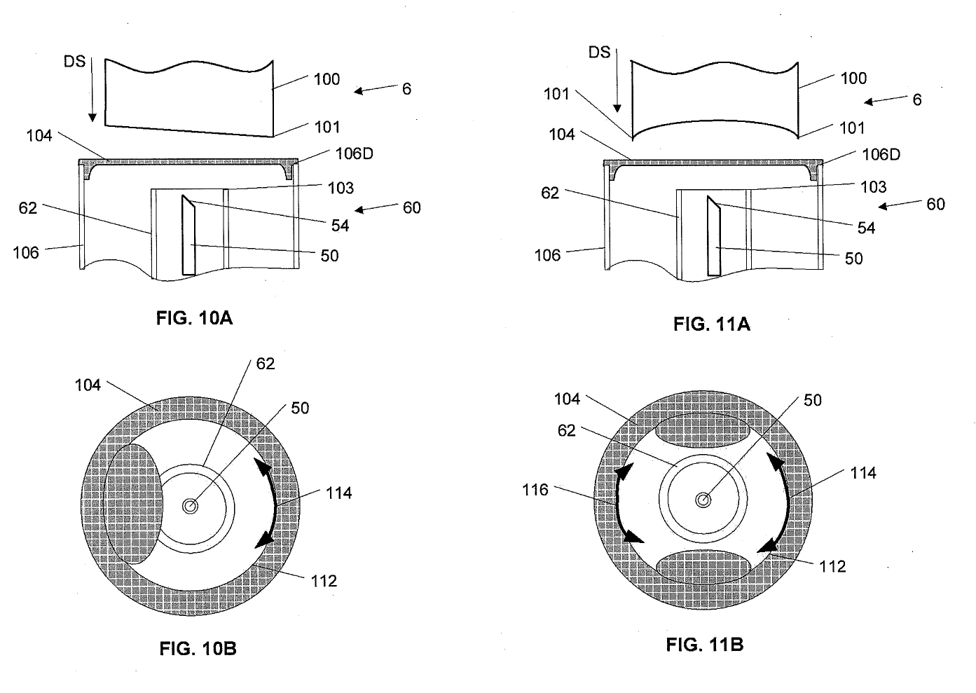

FIGS. 10A-B depict an example of the disabling of the conduit sterility cap 104. FIG. 10A depicts a side cross-sectional view of a portion of cartridge 6 and conduit assembly 60. According to FIG. 10A, conduit assembly 60 includes cartridge-receiving conduit 50 with fluid inlet 54. Cartridge-receiving conduit 50 can be axially surrounded by sleeve 62. Cartridge-receiving conduit 50 and sleeve 62 are surrounded by conduit housing 106 having a distal end 106D upon which is disposed the sterility cap 104. Cartridge 6 includes casing 100 having a conduit sterility cap disabling portion or feature 101 that forms a leading portion 101 of casing 100 relative to displacement direction DS. Casing 100 is moving in displacement direction DS such that disabling portion 101 is about to disable sterility cap 104.

FIG. 10B depicts a top view in which sterility cap 104 includes a score pattern 112 that cause a preferential tear pattern when sterility cap 104 is punctured by disabling portion 101. According to 10B, sterility cap 104 is being disabled by portion 101 from a single location 114 such that a tear in cap 104 is propagating according to arrows leaving location 114 under the influence of a score pattern. This allows sterility cap to move out of the way and prevent contamination of the coupling between needle tip 54 and plunger 48.

FIGS. 11A-B depict a conduit sterility cap disabling feature 101 for which there are two leading features 101 upon casing 100. This results in two locations 114 and 116 from which the tear in cap 104 propagates. Except for the difference in the feature 101 and tear pattern (FIG. 11B) all other elements of FIGS. 11A-B are the same as FIGS. 10A-B. Disabling feature 101 can also be three or more disabling features that are optimized for a given sterility cap design.

Various disabling portions 101 can be envisioned that will rupture and initiate tear patterns upon sterility cap 104 that prevents contamination of the coupling between needle tip 54 and plunger 48.

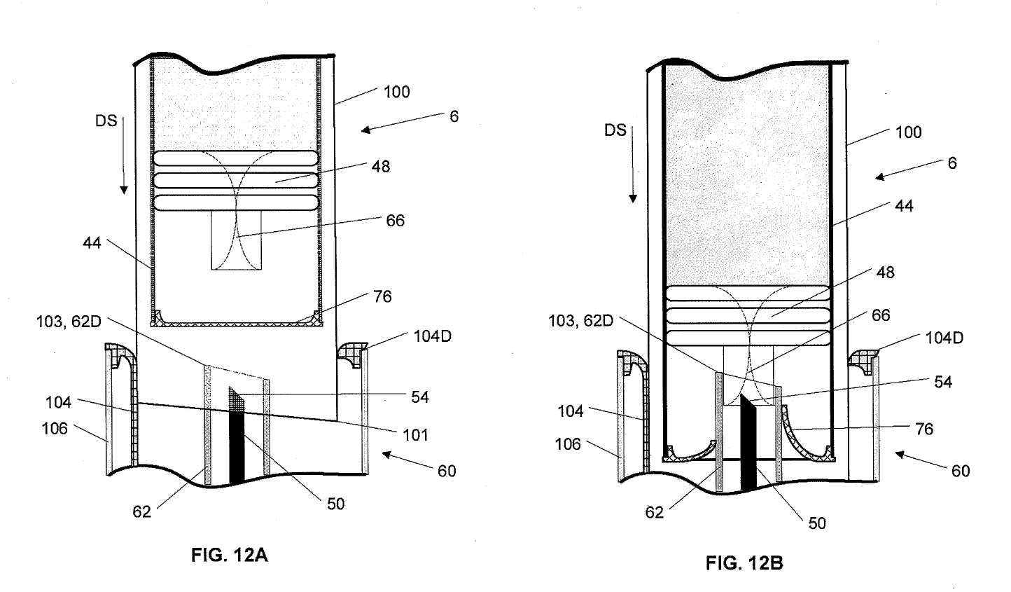

FIGS. 12A-B are cross sectional views depicting an example of the disabling of cartridge sterility cap 76 after sterility cap 104 is already disabled. Cartridge 6 includes casing 100 containing slideably disposed plunger 48 having fluid outlet 66. Sterility cap 76 is disposed within casing 100 and recessed back from leading surface 101 of casing 100.

According to FIG. 12A, sleeve 62 includes cartridge sterility cap disabling feature 103 that is about to impinge upon cartridge sterility cap 76 as casing 100 moves or translates in displacement direction DS. Disabling feature 103 defines a leading or distal end 62D of sleeve 62. It is to be understood that there can be two or more disabling features at the distal end 62D of sleeve 62. In addition, distal end 62D of sleeve 62 is configured to engage a portion 118 of plunger 48 surrounding fluid outlet 66 to align fluid outlet 66 and fluid inlet 54. Distal end 62D can have a tapering surface to facilitate alignment between portion 118 and distal end 62D.

According to FIG. 12B, feature 103 has disabled cap 76 and needle tip 54 is coupling to fluid outlet 66. According to FIG. 12B caps 76 and 104 have been disabled sequentially so as to enable fluid coupling between fluid outlet 66 and fluid inlet 54 without external or cap induced contamination. Each of sterility caps 76 and 104 have been disabled automatically as a result of the displacement of casing 100 in displacement direction DS.

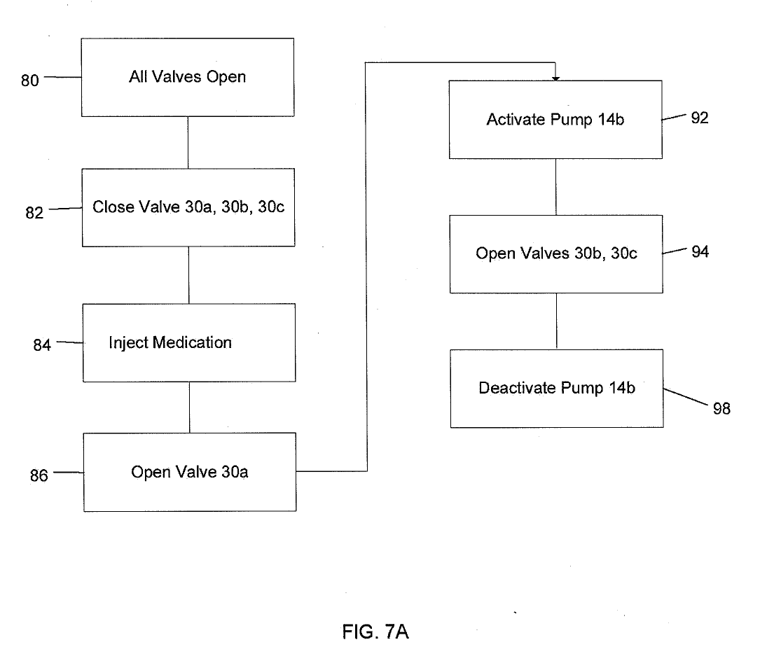

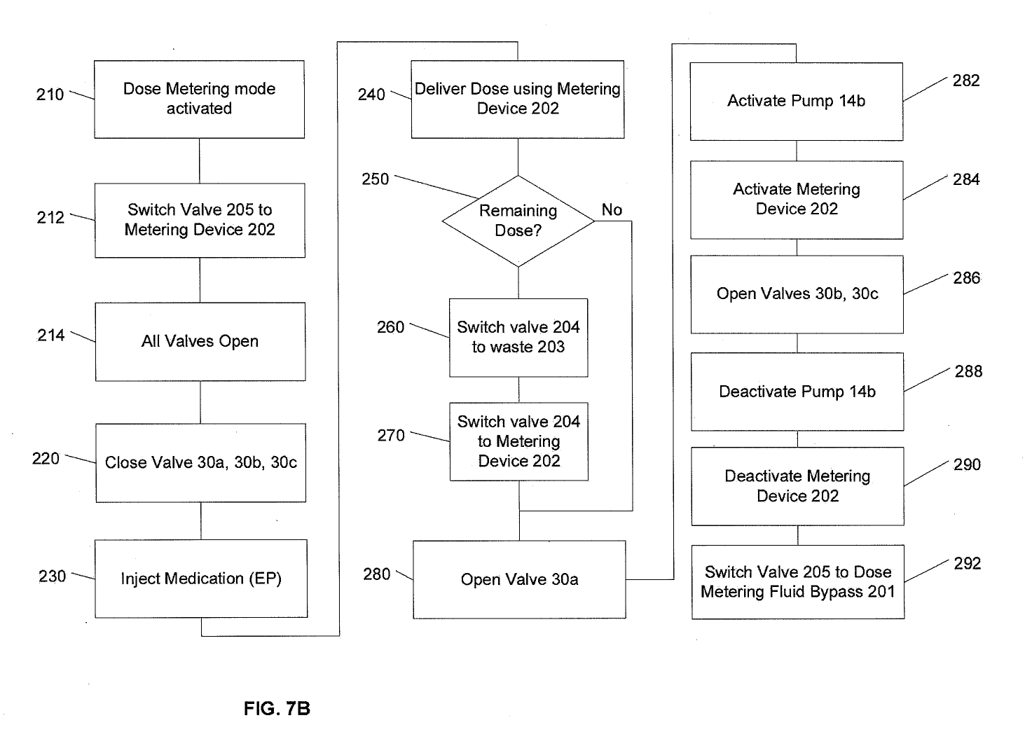

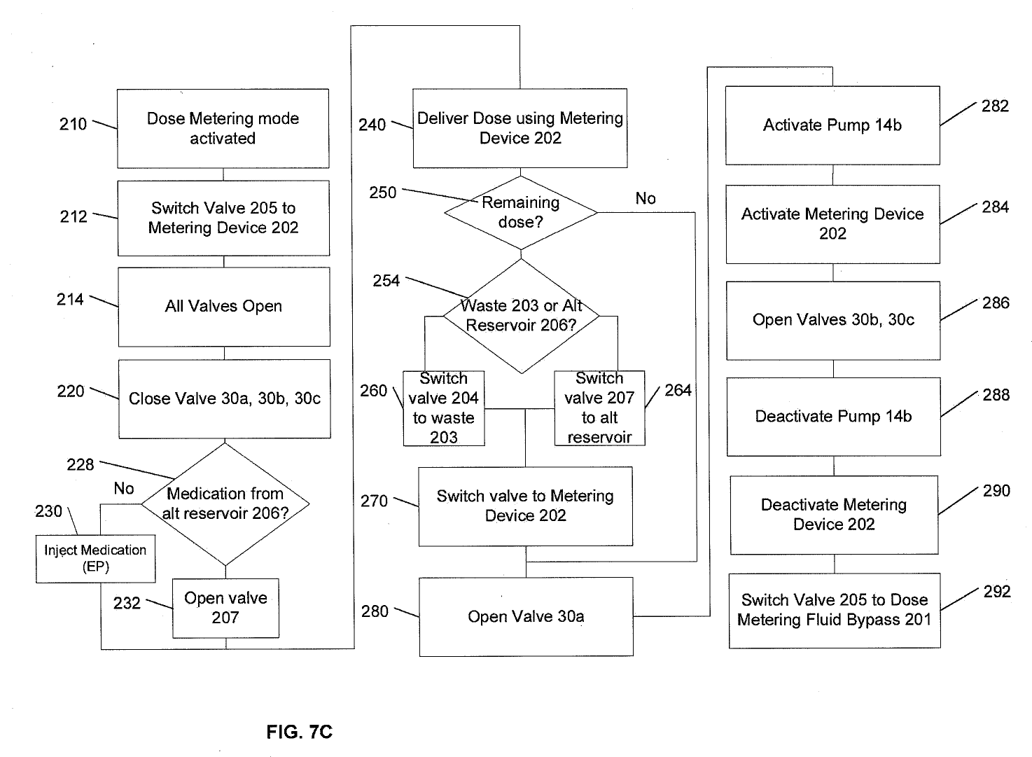

FIG. 7A depicts in flow chart form a method of delivering a medication to a patient. During system 2 operation and following a conduit priming procedure, according to 80, valves 30a-30c are all open and fluid is being delivered from external fluid source 12 (FIG. 1) to a patient, for example through an established IV or IO line. A request for medication is made, for example by a user through a user interface 24 and controller 20. According to 82, valves 30a, 30b and 30c (FIG. 2A) are closed to prevent backflow and restrict flow to only the downstream direction (towards the patient) while medication is delivered from cartridge 6 in 84. Valve 28 can be closed to resist backflow towards the external fluid source 12. According to 84, a cartridge 6 containing a medication is injected into medication-receiving conduit 4a once controller 20 activates cartridge 6 release mechanism 74. Cartridge 6 can be injected according to the sequences described herein.

According to 86, valve 30a is then opened to selectively allow fluid to push the medication through medication receiving conduit 4a towards the patient (not shown). According to 92, pump 14b is activated while valve 30a is open and valves 30b and 30c remain closed. Pump 14b delivers fluid out of fluid source 14 and through conduit 4a until the medication reaches the patient. According to 94 valves 30b and 30c are then opened. Pump 14b is deactivated at about the same time valves 30b-c are opened according to 98.

FIG. 7B depicts another method in flow chart form wherein a prescribed amount of medication is delivered to a patient according to a metering mode discussed for example with respect to FIG. 2B. For illustrative purposes the delivery of a metered dose of epinephrine will be described but it is to be understood that this discussion may apply to any medication from any cartridge 6. According to 210, the Dosage Metering System 200 is activated and a metered prescribed amount of epinephrine to be delivered is released into conduit 4. Valve 205 is actuated to enable fluid to be metered to output conduit 16 according to 212 using dosage metering device 202.

According to 214, valves 30a-30c are all open and fluid is being delivered from external fluid source 12 (FIG. 1) to a patient, for example through an established IV or IO line. A request for epinephrine can be made by the user through a user interface 24 and controller 20. According to 220, valves 30a, 30b and 30c (FIG. 2B) are closed to prevent backflow and restrict flow to only the downstream direction (towards the patient) while medication is delivered from cartridge 6 in 230. Valve 28 also acts to resist backflow towards the external fluid source 12. According to 230, a cartridge 6 containing epinephrine is activated by controller 20 and release mechanism 74 so that it injects medication into medication-receiving conduit 4a. The medication from cartridge 6 may be injected according to the sequences described herein.

According to 240, dosage metering device 202 meters fluid received from medication receiving conduits 4 to output conduit 16 pursuant to the prescribed amount of medication to be delivered from cartridge 6 to the patient. Controller 20 can determine the amount of medication to be delivered based on the type of patient (e.g. adult or pediatric) and the necessity for a time metered dosage determined by the medication selected by the user. According to 250, a decision is made whether any remaining medication is present; otherwise action is moved to 280. For remaining medication (e.g. the full bolus of medication minus the prescribed amount) to be disposed from cartridge 6, valve 204 is opened directing remaining medication through conduit 208 to waste container 203 according to 260. After the remaining medication has been transferred to waste container 203, valve 204 is closed according to 270.

According to 280, valve 30a is then opened to selectively allow fluid to push the epinephrine through medication receiving conduit 4a. According to 282, pump 14b is activated while valve 30a is open and valves 30b and 30c remain closed. Pump 14b delivers fluid out of fluid source 14 and through conduit 4a. According to 284, the metering device delivers fluid until the prescribed medication has reached the patient. According to 286 valves 30b and 30c are then opened. Pump 14b and metering device 202 are both deactivated at about the same time valves 30b-c are opened according to 288 and metering device 202 is deactivated according to 290. According to 292, metering device valve 205 is switched to fluid bypass.

FIG. 7C depicts another method in flow chart form wherein a dosed or prescribed amount of medication such as epinephrine is delivered to a patient according to the metering mode discussed for example with respect to FIG. 2C. For illustrative purposes the delivery of a metered dose of epinephrine will be described but it is to be understood that this discussion may apply to any medication from any cartridge 6. According to 210, the dosage metering system 200 is activated and a prescribed metered amount of epinephrine to be delivered is selected. Valve 205 is actuated to enable fluid to be metered to output conduit 16 according to 212 using dosage metering device 202.

According to 214, valves 30a-30c are all open and fluid is being delivered from external fluid source 12 (FIG. 1) to a patient, for example through an established IV or IO line. A request for epinephrine is made. According to 220, valves 30a, 30b and 30c (FIG. 2C) are closed to prevent backflow and restrict flow to only the downstream direction (towards the patient) while medication is delivered. Valve 28 also acts to resist backflow towards the external fluid source 12. According to 228 a decision to use medication from an alternate reservoir 206 or a cartridge 6 containing epinephrine is made so that it injects medication into medication-receiving conduit 4a. The medication from cartridge 6 may be injected according to the sequences described herein. According to 232 alternate reservoir 206 may be accessed through conduit 209 for using previously filled medication after opening valve 207.

According to 240, a metered dose of dispensed cartridge 6 or alternate reservoir 206, medication is delivered to patient. According to 250, a decision is made whether any remaining medication is present; otherwise action is moved to 280. For remaining medication (e.g. the full bolus of medication minus the prescribed amount) to be disposed from cartridge 6 a decision is made according to 254, to open valve 204 or valve 207 to dispose remaining fluid through conduit 208 to waste 203 or through conduit 209 to alternate reservoir 206 for later use. If remaining fluid is sent to waste, then valve 204 is opened according to 260 or alternately valve 207 is opened to alternate reservoir 206 according to 264. Upon cartridge 6 being fully expelled or alternate reservoir being emptied to a metered amount, valve 204 or 207 is closed according to 270.

According to 280, valve 30a is then opened to selectively allow fluid to push the epinephrine through medication receiving conduit 4a. According to 282, pump 14b is activated while valve 30a is open and valves 30b and 30c remain closed. Pump 14b delivers fluid out of fluid source 14 and through conduit 4a. According to 284, the metering device delivers fluid until the prescribed medication has reached the patient. According to 286 valves 30b and 30c are then opened. Pump 14b and metering device 202 are both deactivated at about the same time valves 30b-c are opened according to 288 and 290. According to 292, metering device valve 205 is switched to fluid bypass. Many variations of the sequence of events can be considered to allow fluids to be partially dispensed with the balance going to an alternate reservoir for future use. It can also be envisioned that a plurality of alternate reservoirs could be configured to do the same.

It should be appreciated that variations of the medical delivery systems described herein can exist. For example, the branching configuration of medication-receiving conduits 4 can have more or less than three parallel conduits 4. Orientation of the cartridges can vary as can the arrangement of cartridges within the cassettes. Cartridges can also be used in the absence of a cassette. As another example, fluid supply 14 can incorporate a “bag squeezer,” or other type of pump rather than a peristaltic pump in order to pressurize the fluid solution. It should also be appreciated that a variety of elements described herein can be used individually or in a variety of combinations. For example, the sterility barrier assemblies described above with respect to the cartridges and cartridge-receiving ports can be optional features that are not necessarily integral to the medication delivery system as a whole. The sterility barriers can be used with fluid injection systems separate from the medication delivery system described herein. Features described herein in the context with or respect to one exemplary device or system can be implemented separately or in any suitable sub-combination with other exemplary devices or systems.

It should also be appreciated that the systems described herein can be used manually or via electronic automation. Although the medication delivery system is described herein in terms of emergency use such as during an ACLS protocol, the medication delivery system can be used for other medication delivery protocols. Also, the delivery system can be used with various routes of administration including intravenous as well as other routes of administration such as an intraosseous route of administration, for example, in the event of a venous access problem.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

What is claimed is:

1. A medication delivery system comprising:
an array of cartridge-receiving ports, each port comprising a fluid channel terminating at a fluid outlet, each port for receiving one of a plurality of cartridges, each cartridge containing a bolus volume of medication;
a medication-receiving fluid conduit fluidically coupled to the fluid outlet of each cartridge-receiving port at a first region and to a system outlet conduit at a second region, wherein the system outlet conduit is adapted to be coupled to a patient catheter line; and
a plurality of dispensing elements, each dispensing element corresponding to a single cartridge-receiving port, wherein upon activation of the dispensing element at least a portion of the bolus volume of medication flows through the fluid outlet of each cartridge-receiving port into the medication-receiving fluid conduit.

2. The system of claim 1, further comprising a plurality of cartridges, each cartridge comprising a hollow outer casing having a proximal end and a distal end with an internal cavity extending therebetween, a plunger, wherein the bolus volume of medication is contained within the internal cavity of the casing.

3. The system of claim 2, wherein the outer casing of each cartridge is translatable in a first direction toward each cartridge-receiving port.

4. The system of claim 3, further comprising a sleeve axially disposed within each cartridge-receiving port, wherein the sleeve applies a restraining force on the plunger in a second direction opposite of the first direction upon translation of the outer casing in the first direction, and wherein translation of the outer casing in the first direction and restraint of the plunger from translation in the first direction reduces a volume of the internal cavity.

5. The system of claim 3, wherein each dispensing element translates the outer casing of each cartridge in the first direction.

6. The system of claim 5, wherein the dispensing element comprises a compressed spring, a source of compressed air, or a push rod.

7. The system of claim 2, wherein the plunger is located near the proximal end of the casing and a distance away from the distal end of the casing, and wherein the bolus volume of medication is disposed inside the internal cavity between the plunger and the distal end of the casing.

8. The system of claim 7, wherein each dispensing element translates the plunger of each cartridge in the first direction toward the distal end of the casing.

9. The system of claim 8, wherein each dispensing element comprises a compressed spring, a source of compressed air, or a push rod.

10. The system of claim 2, further comprising a plurality of activation elements, each activation element being mechanically associated with a single dispensing element and when activated, triggers the dispensing element to dispense at least a portion of the bolus volume of medication from the internal cavity.

11. The system of claim 10, wherein each dispensing element comprises a compressed spring, a source of compressed air, or a push rod.

12. The system of claim 10, further comprising a controller wherein the plurality of activation elements are selectively activated by the controller to trigger the dispensing elements to dispense at least a portion of the bolus volume of medication from the internal cavity.

13. The system of claim 2, wherein at least a portion of each of the plurality of cartridges are disposed within a portable first cassette, wherein the first cassette fluidically couples each of the cartridges to a single cartridge-receiving port upon installation of the first cassette into the system.

14. The system of claim 13, wherein the first cassette comprises a trailing end and a leading fluid delivery end relative to a direction of installation into the system, wherein the trailing end is positioned higher than the leading fluid end when the first cassette fluidically couples each of the cartridges to the single cartridge-receiving port.

15. The system of claim 13, wherein the first cassette comprises dimensions defined along three mutually perpendicular axes including a columnar axis, a transverse axis, and a displacement direction axis, wherein each cartridge is disposed within the first cassette along the columnar axis of the first cassette.

16. The system of claim 15, further comprising a plurality of medication-receiving fluid conduits fluidically coupled to a plurality of cartridge-receiving ports.

17. The system of claim 16, wherein each of the plurality of medication-receiving fluid conduits is fluidically in parallel with one another.

18. The system of claim 17, wherein the plurality of cartridges is disposed within a plurality of cassettes, wherein the plurality of cassettes fluidically couple each of the cartridges to a single cartridge-receiving port upon installation of the plurality of cassettes into the system.

19. The system of claim 18, wherein each of the plurality of cassettes is installed into the system in a side-by-side configuration such that the cassettes are compactly arranged in a housing of the system along the transverse axis.

20. The system of claim 16, further comprising a fluid supply line to deliver a pressurized supply of fluid from a fluid source to the first and second medication-receiving conduits.

21. The system of claim 20, wherein the plurality of medication-receiving conduits each comprise a one-way valve, wherein the valve controls forward and backward fluid flow facilitating selective flushing of each medication-receiving conduit.

22. The system of claim 2, wherein the plunger of each cartridge comprises a fluid outlet and each cartridge-receiving port further comprises a fluid inlet.

23. The system of claim 22, wherein each cartridge fluid outlet fluidically couples to each port fluid inlet such that the bolus volume of medication is in fluid communication with the medication-receiving fluid conduit, and wherein each dispensing element, when activated, urges at least a portion of the bolus volume of medication to flow into the medication-receiving fluid conduit toward the system outlet conduit.

24. The system of claim 2, further comprising a one-way valve disposed in each cartridge-receiving port, wherein the valve allows flow from each cartridge into the medication-receiving conduit and prevents flow from the medication-receiving conduit into each cartridge.

25. The system of claim 1, further comprising a fluid supply line to deliver a pressurized supply of fluid from a fluid source to the medication-receiving conduit and a one-way valve within the fluid supply line to prevent flow of the supply of fluid back into the fluid source.

26. The system of claim 2, further comprising a plurality of sensors disposed within each cartridge-receiving port, wherein each sensor detects status information of a single corresponding cartridge, the status information detected selected from the group comprising presence of each cartridge coupled to a single port, proper coupling of a cartridge to a single port, volume of medication within each cartridge, and type of medication within each cartridge.

27. The system of claim 13, further comprising a sensor disposed within a housing of the system, wherein the sensor detects status information of the first cassette upon installation of the first cassette into the system, the status information detected selected from the group comprising presence of the cassette installed into the system, proper installation of a cassette, and presence of a cartridge disposed within the cassette.

28. The system of claim 2, wherein the volume of medication is selected from the group consisting of epinephrine, lidocaine, amiodarone, atropine, magnesium sulfate, naloxone, adenosine, sodium bicarbonate, dextrose, vasopressin, and calcium chloride.

29. The system of claim 28, wherein the system delivers sequential volumes of medication to a patient according to an advanced cardiac life support protocol.

30. The system of claim 2, further comprising a plurality of medication-receiving fluid conduits fluidically coupled to the fluid outlet of each cartridge-receiving port, wherein the plurality of cartridges are fluidically coupled to the plurality of medication-receiving fluid conduits in at least two dimensions.

31. The system of claim 2, further comprising a fluid flow metering device disposed within the medication-receiving fluid conduit between the first region and the system outlet conduit.

32. The system of claim 31, wherein the fluid flow metering device further comprises a controller selectable between a bolus mode and a controlled mode that controls the flow of the volume of medication through the system outlet conduit.

33. The system of claim 32, wherein the bolus mode delivers all of the bolus volume of medication contained within the internal cavity to the system outlet conduit and the controlled mode delivers a volume of medication to the system outlet conduit that is less than the bolus volume of medication contained within the internal cavity of the casing.

34. A medication delivery system comprising:
a medication-receiving fluid conduit comprising a plurality of medication-receiving ports to fluidically communicate with a corresponding plurality of cartridges, each cartridge containing a bolus volume of medication;
a metering system comprising an inlet port in fluid communication with the medication-receiving fluid conduit; an outlet port in fluid communication with a patient catheter line; and a dosage metering device; and
a controller selectively operable in one of two modes including a controlled mode wherein the metering system meters a prescribed portion of the bolus volume of medication to the outlet port; and a full bolus mode wherein the metering system meters the bolus volume of medication to the outlet port.

35. The system of claim 34, wherein the metering system further comprising a first conduit positioned between the inlet port and the outlet port.

36. The system of claim 35, wherein the metering system operable in the controlled mode delivers a first portion of the bolus volume of medication through the medication-receiving fluid conduit to the outlet port and a second portion of the bolus volume of medication to the first conduit.

37. The system of claim 36, wherein the first portion is delivered to the outlet port after the second portion is delivered to the first conduit.

38. The system of claim 36, wherein the first conduit terminates at a waste container or an alternate reservoir.

39. The system of claim 34, further comprising a plurality of cartridges, wherein each cartridge comprises a hollow outer casing having a proximal end and a distal end with an internal cavity extending therebetween, a plunger, and a bolus volume of medication contained within the internal cavity of the casing.

40. The system of claim 39, wherein the medication-receiving fluid conduit fluidically couples to the plurality of cartridges through corresponding medication-receiving ports.

41. A medication delivery method comprising:
providing a system comprising a medication-receiving fluid conduit comprising a plurality of cartridge-receiving inlet ports at a first region and a fluid outlet at a second region, wherein the ports are mechanically supported on a rigid base in a housing of the system;
installing into the system a cassette containing a plurality of cartridges each having a volume of medication disposed therein, wherein installing the cassette couples each cartridge with a single cartridge-receiving inlet port;
coupling the fluid outlet to a patient catheter line;
activating a dispensing element corresponding to at least one of the plurality of cartridges; and
displacing at least a portion of the volume of medication from that at least one cartridge, the volume of medication flowing into the medication-receiving fluid conduit and through the fluid outlet.
